(19) United States
(12) Patent Application Publication
(10) Pub. No.: US 2007/0123452 A1
(43) Pub. Date: May 31, 2007

(54) Method of selecting novel immunosuppressant

(75) Inventors: Hideaki Matsuoka, Tokyo (JP); Takao Fujimura, Tokyo (JP); Masako Hayashi, Tokyo (JP); Ichiro Aramori, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Astellas Pharma Inc., Tokyo (JP)

(21) Appl. No.: 10/540,546

(22) PCT Filed: Dec. 26, 2003
(86) PCT No.: PCT/JP03/16895

§ 371 Date: Jun. 24, 2005

(30) Foreign Application Priority Data
Dec. 27, 2002 (JP) 2002-378800

(51) Int. Cl. A61K 48/00; A61K 38/12; A61K 31/19; C12Q 1/34

(52) U.S. Cl. 514/9; 514/44; 514/575; 435/18

(57) ABSTRACT

The present invention provides a method of selecting an IL-2 production inhibitor and/or an immunocyte proliferation inhibitor having low GATA-1 production inhibitory activity, which comprises measuring the HDAC4 and/or HDAC8 inhibitory activity of a test substance, and a method of selecting an immunosuppressant having low thrombocytopenic activity, which comprises measuring the HDAC4 and/or HDAC8 inhibitory activity of a test HDAC inhibitor.

### FIELD OF THE INVENTION

The present invention relates to a method of selecting an immunosuppressant having low thrombocytopenic activity, and an IL-2 production inhibitor and/or immunocyte proliferation inhibitor having low GATA-1 production inhibitory activity, which selectively suppress HDAC4 and/or HDAC8 only, as well as an agent for suppressing immunity having low thrombocytopenic activity, and an agent for inhibiting IL-2 production and/or an agent for inhibiting immunocyte proliferation having low GATA-1 production inhibitory activity, which are obtained by the method, a kit used for the method, and the like.

### BACKGROUND ART

Cyclosporin A (CsA) and tacrolimus (FK506), which are major immunosuppressants that are today widely used in clinical settings to suppress acute graft rejection reactions following organ transplantation, inhibit the activity of calcineurin, which is a Ca²⁺/calmodulin-dependent protein phosphatase, by binding to respective specific immunophilins (for example, cyclophilin for CsA, and FKBP12 for FK506). It is known that as a result, the dephosphorylation reaction of NF-AT (Nuclear Factor of Activated T-cell) is inhibited, which in turn inhibits the transfer of NF-AT into nucleus and suppresses the transcriptional activity of the IL-2 gene. Research into this action mechanism has shown that the specific suppression of the expression of the IL-2 gene at the transcription level in activated T-cells is of paramount importance to the suppression of organ graft rejection and the therapeutic efficacy for various autoimmune diseases and the like.

The cell nuclear DNA of eukaryotic organisms has a chromatin structure with nucleosome as the structural unit, and is folded in strata. Each nucleosome comprises two molecules of each of the H2A, H2B, H3 and H4 histones, and one molecule of the H1 histone. A particular lysine residue present on the N-terminal side of these histone proteins is known to often undergo modification by acetylation and to be associated with the expression regulation of various genes.

The level of the acetylation on the histone N-terminal side is exquisitely controlled in a balance of the activities of two mutually counteracting series of enzymes, that is, histone acetyltransferase (histone acetylation enzyme, HAT) and histone deacetylase (histone deacetylation enzyme, hereinafter abbreviated HDAC as required). Of these enzymes, histone deacetylase, a metal enzyme having zinc coordinated at the active center thereof, is responsible for the function of removing the acetyl group from acetylated histone; this acetyl group removal is considered to cause aggregation of the chromatin structure and lead to suppressed transcription.

A series of studies have shown that human histone deacetylase occurs in at least 11 kinds of isoforms, from HDAC1 to HDAC11 (see, for example, Pandey R et al., Nucleic Acids Research, 30(23): 5036-55 (2002); L. Gao et al., Journal of Biological Chemistry, 277(28): 25748-25755 (2002); Joseph J. BUGGY et al., Biochemical Journal, 350: 199-205 (2000); International Patent Publication No. WO00/10583). As such, each isoform has been shown not to function as a single enzyme, but to interact with many other proteins to form a larger complex. Of these HDAC isoforms, HDAC1, HDAC2, HDAC3 and HDAC8 are structurally highly homologous to the yeast Rpd3 protein, and are classified under class I. HDAC1 and HDAC2 are components of a complex comprising a large number of subunit structures, and are found in, for example, the SIN3 complex and the NURD/Mi2 complex. HDAC3 is known to form a complex with N-CoR (nuclear receptor corepressor) or SMRT (silencing mediator of retinoid and thyroid) and mediate the transcription suppression by, for example, thyroid hormone receptor (TR) or v-ErbA. On the other hand, a group of enzymes that are homologous to the yeast Hdal protein are classified under class II, and HDAC4, HDAC5, HDAC6 and HDAC7 are known to belong to this group. HDAC4, HDAC5 and HDAC7, which are class II histone deacetylases, are known to be capable of interacting with BCoR, which is a supplementary co-repressor that mediates the suppression by N-COR, SMRT or BCL-6.

Although the recombinant HDAC3 itself is inactive as a single substance, data suggestive of its capability of functioning as an active enzyme with the addition of SMRT or N-COR have been reported (see, for example, Matthew G. Guenther et al., Molecular and Cellular Biology, 21(18): 6091-6101 (2001)), and HDAC4, which is a class II enzyme, has been reported to interact with HDAC3, which is a class I enzyme, via SMRT or N-COR in vivo (see, for example, Wolfgang Fischie et al., Molecular Cell, 9: 45-57 (2002)). However, much remains unknown concerning the accurate mechanism by which these HDAC isoforms are activated.

To date, extensive screening has discovered a large number of compounds that have HDAC inhibitory activity, including compounds that have remarkable IL-2 production inhibitory activity (see, for example, I. Takahashi et al., The Journal of Antibiotics, 49: 453-457 (1995)), which are attracting attention as candidates for immunosuppressants that supplement cyclosporin and tacrolimus. In fact, among the compounds thus selected, those that exhibit excellent immunosuppressive action in vivo have been found. FR225497 has been found to be excellently effective as a therapeutic agent or prophylactic agent for organ transplant rejection and autoimmune diseases with its potent immunosuppressive action (see, for example, International Patent Publication No. WO00/08048) and, in addition, has been suggested to be useful as a therapeutic agent or prophylactic agent for many other diseases considered to develop due to an abnormality in gene expression. Examples of such diseases include inflammatory disorders, diabetes, diabetic complications, homozygous thalassemia, fibrosis, liver cirrhosis, acute promyelocytic leukemia (APL), protozoal infection, cancers (tumors) and the like. However, despite this utility, many of these compounds that have HDAC inhibitory activity pose the problem of being likely to cause serious thrombocytopenia as a side effect when administered to the body, and this aspect has made it difficult to use such compounds as actual therapeutic drugs.

The reason why many of immunosuppressive HDAC inhibitors are likely to cause thrombocytopenia as a side effect has not yet fully been elucidated. However, the present inventors previously showed that the suppression of the expression of the gene for GATA-1 (also called GATA-1 binding protein, GF-1, NF-El or Eryf 1) is profoundly involved in the thrombocytopenic activity of HDAC inhibitors as a serious side effect, and that compounds with more potent platelet suppressive action tend to more strongly suppress the transcription of the GATA-1 gene (see Japanese Patent Application No. 2002-203901).

GATA-1 is a DNA-binding protein that recognizes the (A/T)GATA(A/G) consensus sequence, which is characteristically present in the transcription control regions of hematopoietic genes. This GATA motif sequence is found in a variety of control regions such as enhancer regions of various globin genes, locus control regions (LCRs) of the β-globin gene, enhancer regions of the T-cell receptor α-chain and δ-chain genes, and in promoters. Also, GATA-1 mRNA is expressed at high levels in mature erythrocytes, mast cells, megakaryocyte and the like, with slight expression observed in multifunctional precursor cells and juvenile mouse testis. Furthermore, mice having the GATA-1 gene knocked out by an ordinary method are lethal due to primary hematopoietic cell hypoplasia in the stage of embryogenesis (see, for example, Y. Fujiwara et al., Proceedings of the National Academy of Sciences of the USA, 93: 12355-12358 (1996)). On the other hand, it is possible to prepare mice having only the expression of the GATA-1 gene in megakaryocytic lineage knocked out selectively, and these mice have been shown to suffer dramatically decreased platelet counts and to lack normal maturation of megakaryocytic lineage (see, for example, R. A. Shivdasani et al. The EMBO Journal, 16: 3965-3973 (1997)).

In HDAC, a large number of isozymes exist, some of which have a side effect serious to the body when inhibited. For example, administering a conventional HDAC inhibitor, which has no isozyme selectivity, causes thrombocytopenia as a side effect, and the present inventors showed that this action is possibly attributable to the transcription suppressive action of the GATA-1 gene, which plays a decisive role in the differentiation and maturation of erythrocytic lineage cells and megakaryocytic lineage, and provided a method of screening for an immunosuppressant having low thrombocytopenic activity based on this action (see Japanese Patent Application No. 2002-203901). However, in this screening method, to screen for an immunosuppressant having low thrombocytopenic activity, it is necessary to separately evaluate the platelet suppressive action of a compound possessing HDAC inhibitory action after the compound is screened for, so that much labor and time are taken and the screening method is not always satisfactory. Accordingly, to select an immunosuppressant from among HDAC inhibitors, there is a strong demand for the provision of an excellent method of screening for an immunosuppressant that inhibits a particular isozyme involved in immunosuppressive effects, but does not inhibit other isozymes involved in side effects such as thrombocytopenic activity, conveniently and in a short time, and the present invention is directed to resolve these problems.

### DISCLOSURE OF THE INVENTION

The present inventors diligently studied the association of the transcription suppressive activities for the IL-2 gene and the GATA-1 gene and various HDACs, and found that if either or both of HDAC4 or HDAC8 out of the various HDACs, are selectively suppressed, IL-2 production can be inhibited without noticeably suppressing GATA-1 production inhibitory activity, the proliferation of immunocytes can be inhibited, and immunity can be suppressed without significantly decreasing the platelet count. The present inventors confirmed that HDAC4 forms a complex with N-CoR, and the complex of HDAC4 and N-CoR further forms a complex with HDAC3. Accordingly, the present inventors realized that an IL-2 production inhibitor having low GATA-1 production inhibitory activity, an immunocyte proliferation inhibitor, or an immunosuppressant having low thrombocytopenic activity, can be obtained by selecting a compound that selectively suppresses HDAC4 and/or HDAC8 out of various HDACs, and inhibiting the formation of a complex of HDAC4 and N-CoR and, as required, the formation of a higher complex of HDAC4 and the N-CoR-HDAC3 complex, and developed the present invention.

Accordingly, the present invention relates to the inventions shown below.
[1] an agent for inhibiting IL-2 production and/or an agent for inhibiting immunocyte proliferation which have low GATA-1 production inhibitory activity, comprising a selective HDAC4 and/or HDAC8 inhibitor.
[2] an agent for suppressing immunity having low thrombocytopenic activity, comprising a selective HDAC4 and/or HDAC8 inhibitor.
[3] a method of selecting an IL-2 production inhibitor and/or an immunocyte proliferation inhibitor which have low GATA-1 production inhibitory activity, which comprises measuring the HDAC4 and/or HDAC8 enzyme inhibitory activity of a test substance.
[4] a method of selecting an immunosuppressant having low thrombocytopenic activity, which comprises measuring the HDAC4 and/or HDAC8 enzyme inhibitory activity of a test substance.
[5] a method of selecting an immunosuppressant having low thrombocytopenic activity, which comprises steps (i) and (ii) below:
(i) measuring the HDAC4 and/or HDAC8 enzyme inhibitory activity of a test substance, and
(ii) measuring the HDAC enzyme inhibitory activity of the test substance, wherein the HDAC enzyme inhibitory activity is one or more HDAC enzyme inhibitory activities selected from a group consisting of HDAC1, HDAC2, HDAC3, HDAC5, HDAC6 and HDAC7 enzyme inhibitory activities.
[6] the method described in [3], which is a method of selecting an IL-2 production inhibitor and/or an immunocyte proliferation inhibitor which have low GATA-1 production inhibitory activity, and which comprises steps (i) to (iii) below, wherein the IL-2 production inhibitor and/or the immunocyte proliferation inhibitor selectively inhibits HDAC4 and/or HDAC8 enzyme activity:
(i) expressing each of the HDAC1, HDAC2, HDAC3, HDAC4, HDAC5, HDAC6, HDAC7 and HDAC8 genes to obtain respective enzyme solutions;
(ii) measuring HDAC enzyme activity for each of the enzyme solutions in the presence of a test substance;
(iii) selecting a test substance that selectively suppresses HDAC enzyme activity only when using an enzyme solution obtained by expressing the HDAC4 gene or the HDAC8 gene.
[7] the method described in [4], which is a method of selecting an immunosuppressant having low thrombocytopenic activity, and which comprises steps (i) to (iii) below, wherein the immunosuppressant selectively inhibits HDAC4 and/or HDAC8 enzyme activity:
(i) expressing each of the HDAC1, HDAC2, HDAC3, HDAC4, HDAC5, HDAC6, HDAC7 and HDAC8 genes to obtain respective enzyme solutions;
(ii) measuring HDAC enzyme activity for each of the enzyme solutions in the presence of a test substance;
(iii) selecting a test substance that selectively suppresses HDAC enzyme activity only when using an enzyme solution obtained by expressing the HDAC4 gene or the HDAC8 gene.
[8] the method described in [4], which is a method of selecting an immunosuppressant having low thrombocytopenic activity, and which comprises steps (i) to (iii) below, wherein the immunosuppressant selectively inhibits HDAC4 and/or HDAC8 enzyme activity:
(i) measuring HDAC enzyme activity for an enzyme solution partially purified from human cells, in the presence of a test substance;
(ii) measuring HDAC enzyme activity for an enzyme solution obtained by expressing the HDAC4 gene and/or the HDAC8 gene, in the presence of a test substance;
(iii) selecting a test substance that selectively suppresses HDAC enzyme activity only when using an enzyme solution obtained by expressing the HDAC4 gene and/or the HDAC8 gene, by comparing the enzyme activities measured in (i) and (ii).
[9] a method of selecting an immunosuppressant having low thrombocytopenic activity, which comprises steps (i) to (iii) below, wherein an HDAC inhibitor that specifically inhibits the formation of a complex of HDAC4 and N-CoR is selected from among test HDAC inhibitors:
(i) expressing the HDAC4 gene and the N-CoR gene to obtain an enzyme solution;
(ii) allowing the enzyme solution to coexist with a test HDAC inhibitor;
(iii) determining whether or not a complex of HDAC4 and N-CoR is formed in the mixture of enzyme solution and test HDAC inhibitor obtained in (ii).
[10] a method of selecting an immunosuppressant having low thrombocytopenic activity, which comprises steps (i) and (ii) below, wherein an HDAC inhibitor that specifically inhibits the formation of a complex of the HDAC4 fusion protein comprising at least a portion of the HDAC4 protein, and the N-CoR fusion protein comprising at least a portion of the N-CoR protein, is selected from among test HDAC inhibitors:
(i) expressing the gene that encodes the HDAC4 fusion protein and the gene that encodes the N-CoR fusion protein in cells;
(ii) determining whether or not the test HDAC inhibitor inhibits the formation of a complex of the HDAC4 fusion protein and the N-COR fusion protein.
[11] a method of selecting a compound having low thrombocytopenic activity and having immunosuppressive activity, which comprises steps (i) and (ii) below, wherein an HDAC inhibitor that suppresses the expression level of HDAC4 and/or HDAC8 is selected from among test HDAC inhibitors:
(i) allowing cells that express HDAC4 and/or HDAC8 to coexist with a test HDAC inhibitors;
(ii) measuring the HDAC4 and/or HDAC8 expression level in the cells.
[12] a method of selecting a compound having low thrombocytopenic activity and having immunosuppressive activity, which comprises steps (i) and (ii) below, wherein an HDAC inhibitor that selectively inhibits the binding of HDAC4 and/or HDAC8 and an HDAC4- and/or HDAC8-specific ligand is selected from among test HDAC inhibitors:
(i) expressing the HDAC4 and/or HDAC8 gene to obtain an enzyme solution;
(ii) measuring the binding activity of the HDAC4 and/or HDAC8 enzyme and the HDAC4- and/or HDAC8-specific ligand for the enzyme solution in the presence of an HDAC4- and/or HDAC8-specific ligand and a test HDAC inhibitor.
[13] an assay kit for the selection of an immunosuppressant having low thrombocytopenic activity including a selective HDAC4 and/or HDAC8 inhibitor, which includes at least (i) and (ii) below:
(i) enzyme solutions prepared by expressing each of the HDAC1, HDAC2, HDAC3, HDAC5, HDAC6 and HDAC7 genes;
(ii) an enzyme solution prepared by expressing each of the HDAC4 and/or HDAC8 gene;
[14] a DNA having the base sequence that encodes the amino acid sequence shown by SEQ ID NO:4 or SEQ ID NO:6.
[15] an HDAC4 variant that is the amino acid sequence shown by SEQ ID NO:4 or SEQ ID NO:6.
[16] a method of inhibiting IL-2 production and/or immunocyte proliferation with low GATA-1 production inhibitory activity, which comprises administering an effective amount of selective HDAC4 and/or HDAC8 inhibitor to a subject (for example, any animal, preferably a mammal (for example, human, mouse, rat, rabbit, dog, cat, bovine, horse, goat, sheep and the like), most preferably a human).
[17] a method of suppressing immunity with low thrombocytopenic activity, which comprises administering an effective amount of selective HDAC4 and/or HDAC8 inhibitor to a subject (same as above).
[18] use of a selective HDAC4 and/or HDAC8 inhibitor for the production of an agent for inhibiting IL-2 production and/or an agent for inhibiting immunocyte proliferation which have low GATA-1 production inhibitory activity.
[19] use of a selective HDAC4 and/or HDAC8 inhibitor for the production of an agent for suppressing immunity having low thrombocytopenic activity.

### BRIEF DESCRIPTION OF THE DRAWINGS

FIG. 1 shows the results of a confirmation of the base sequence a 731-basepair fragment in the human IL-2 promoter region, amplified by PCR. The base sequence in the upper panel was obtained from GenBank accession number X00695 (6684 bp in full length), a portion of which (SEQ ID NO:29) was used as a reference control. The base sequence in the lower panel is the base sequence of the IL-2 promoter region in pGL3 IL2 pro prepared in Example 1 (SEQ ID NO:23).

FIG. 2 shows the results of a confirmation of the base sequence a 368-basepair fragment in the human IL-2 promoter region, amplified by PCR. The base sequence in the upper panel was obtained from GenBank accession number X00695 (6684 bp in full length), a portion of which (SEQ ID NO:31) was used as a reference control.

The base sequence in the lower panel is the base sequence of the IL-2 promoter region in pGL3 IL2 pro43 prepared in Example 1 (SEQ ID NO:24).

FIG. 3 shows the results of a confirmation of the base sequence an 821-basepair fragment in the human GATA-1 promoter region, amplified by PCR. The base sequence in the upper panel was obtained from GenBank accession number AF196971 (113853 bp in full length), a portion of which (SEQ ID NO:84) was used as a reference control. The base sequences in the lower panel are the base sequence of the GATA-1 promoter region in pGL3-IE prepared in Example 2, and the base sequence of a portion of the GATA-1 promoter region in pGL3-HSI-IE Pro (SEQ ID NO:25).

FIG. 4 shows the results of a confirmation of the base sequence a 637-basepair fragment in the human GATA-1 promoter region, amplified by PCR. The base sequence in the upper panel was obtained from GenBank accession number AF196971 (113853 bp in full length), a portion of which (SEQ ID NO:85) was used as a reference control. The base sequence in the lower panel is the base sequence of a portion of the GATA-1 promoter region in pGL3-HSI-IE Pro, prepared in Example 2 (SEQ ID NO:26).

FIG. 5 shows the effects of the overexpression of various HDAC isozymes on IL-2 transcriptional activity (upper panel, n=3) and cell proliferation rate (lower panel).

FIG. 6 shows the effects of the overexpression of various HDAC isozymes on GATA-1 transcriptional activity (n=2).

FIG. 7 summarizes the HDAC4 dominant negative variants prepared in Example 6.

FIG. 8 shows IL-2 transcription suppression depending on the expression level of HDAC4 dominant negative variants (H802K, H803L, H863L). The amount of plasmid used for transfection (n=3) is shown on each graph.

FIG. 9 shows the Jurkat-cell-specific cell proliferation inhibition due to the expression of an HDAC4 dominant negative variant (H863L).

FIG. 10 shows the effects of the expression of HDAC4 dominant negative variants (H802K, H803L, H863L) in Jurkat cells and HEL cells on IL-2 and GATA-1 transcription. The upper panel shows the results of an IL-2 reporter gene assay in Jurkat cells for normal HDAC1, HDAC3 and HDAC4, and variants thereof. The lower panel shows the results of a GATA-1 reporter gene assay in HEL cells for normal HDAC1, HDAC3 and HDAC4, and variants thereof.

FIG. 11 shows the expression of HDAC4, HDAC5 and HDAC7 in PEAK Rapid cells (Edge BioSystems), and the formation of a complex of HDAC4, HDAC5 or HDAC7 with N-CoR and HDAC3.

FIG. 12 shows the capability of forming a complex of an HDAC4 dominant negative variant (H863L).

FIG. 13 shows the HDAC activity in wild-type HDAC4 and HDAC4 dominant negative variants (H802K, H803L, H863L).

FIG. 14 summarizes the interaction of HDAC-4 and N-CoR using a two-hybrid system. The upper panel shows the mode of transcription regulation with the interaction of HDAC-4 and N-CoR, and the lower panel shows the mode of transcription regulation without the interaction of HDAC-4 and N-CoR.

FIG. 15 shows the interaction of HDAC-4 and N-CoR using a two-hybrid system.

FIG. 16 shows the effects of HDAC4-specific siRNA on IL-2 and GATA-1 transcription in Jurkat cells and HEL cells. The left panel shows the results of an IL-2 reporter gene assay in Jurkat cells for HDAC4-specific siRNA. The right panel shows the results of a GATA-1 reporter gene assay in HEL cells for HDAC4-specific siRNA.

### DETAILED DESCRIPTION OF THE INVENTION

The present invention is hereinafter described in detail.

The present invention relates to an agent for inhibiting IL-2 production (IL-2 production inhibitor) and/or an agent for inhibiting immunocyte proliferation (immunocyte proliferation inhibitor), each of which having low GATA-1 production inhibitory activity, comprising a selective HDAC4 and/or HDAC8 inhibitor. An IL-2 production inhibitor and/or an immunocyte proliferation inhibitor, each of which having low GATA-1 production inhibitory activity, comprising a selective HDAC4 and/or HDAC8 inhibitor can be obtained by, for example, the selection method described below. It is also possible to obtain an IL-2 production inhibitor and/or an immunocyte proliferation inhibitor, each of which having low GATA-1 production inhibitory activity, comprising a selective HDAC4 and/or HDAC8 inhibitor, by using the selection method described below and a method known in the art in combination.

A “selective HDAC4 and/or HDAC8 inhibitor” refers to an inhibitor that suppresses the HDAC enzyme activity of HDAC4 and/or HDAC8 more than suppresses the HDAC enzyme activity of HDAC1-3 and HDAC5-7, and is preferably an inhibitor that does not or substantially does not suppress the HDAC enzyme activity of HDAC1-3 and HDAC5-7, and that suppresses the HDAC enzyme activity of HDAC4 and/or HDAC8. As examples of the selective HDAC4 and/or HDAC8 inhibitor, an antisense nucleic acid, ribozyme, decoy nucleic acid, siRNA and antibody (for example, single-chain antibody) that are specific for HDAC4 and/or HDAC8, a dominant negative variant of HDAC4 and/or HDAC8, an expression vector carrying a nucleic acid that encodes one of them, and a substance obtained by the selection method described below, can be mentioned.

“GATA-1 production inhibitory activity” may be any activity to suppress GATA-1 production. As examples of the mechanism of suppression of GATA-1 production, suppression at the transcription level, promotion of GATA-1 mRNA decomposition, suppression at the translation level and the like can be mentioned. GATA-1 production inhibitory activity can be evaluated by a method known in the art. Specifically, GATA-1 production inhibitory activity can be evaluated by a reporter assay using the GATA-1 reporter gene as described below, or by measuring the expression level of GATA-1 mRNA or GATA-1 protein in a cell line, primary culture cells and the like that are naturally expressing GATA-1. “Low” GATA-1 production inhibitory activity as evaluated with the addition of a compound means that the activity thereof to inhibit GATA-1 production is lower than that obtained when a compound for a reference control is added; for example, whether or not the GATA-1 production inhibitory activity is low can be evaluated by an assay using the GATA-1 reporter gene. As an example of the compound used as a reference control, tricostatin (TSA), which is a non-specific HDAC inhibitor, can be mentioned.

The “immunocyte proliferation inhibitor” may have any kind of activity, as long as it inhibits the proliferation of immunocytes. As examples of the mechanism of cell proliferation inhibition, cell cycle termination, apoptosis induction and the like can be mentioned. Immunocyte proliferation inhibitory activity can be evaluated by a method known in the art. Specifically, immunocyte proliferation inhibitory activity can be evaluated by thymidine uptake activity, the MTT method and the like. Here, “immunocytes” refer to cells responsible for immune function in the body. Preferably, “immunocytes” refer to lymphocytes, dendritic cells and the like that are capable of produce an antibody or of expressing a cellular immune reaction, more preferably T-cells.

The “IL-2 production inhibitors” may have any kind of activity, as long as it has IL-2 production inhibitory activity, that is, an activity to suppress IL-2 production. As examples of the mechanism of IL-2 production inhibition, suppression at the transcription level, promotion of IL-2 mRNA decomposition, suppression at the translation level and the like can be mentioned. IL-2 production inhibitory activity can be evaluated by a method known in the art. Specifically, IL-2 production inhibitory activity can be evaluated by a reporter assay using the IL-2 reporter gene as described in Examples below, or by measuring the expression level of IL-2 mRNA or IL-2 protein in a cell line, primary culture cells and the like that are naturally expressing the IL-2.

The present invention also relates to an agent for suppressing immunity (immunosuppressant) having low thrombocytopenic activity, comprising a selective HDAC4 and/or HDAC8 inhibitor. An immunosuppressant having low thrombocytopenic activity, comprising a selective HDAC4 and/or HDAC8 inhibitor can be obtained by, for example, the selection method described below. It is also possible to obtain an immunosuppressant having low thrombocytopenic activity, comprising a selective HDAC4 and/or HDAC8 inhibitor, by using the selection method described below and a method known in the art in combination. “Thrombocytopenic activity” refers to an activity to decrease the platelet count, and this thrombocytopenic activity can be directly confirmed by, for example, a method known in the art, such as administering a compound to a laboratory animal. Also, because it is known that there is a correlation between thrombocytopenic activity and GATA-1 production inhibitory activity, the thrombocytopenic activity can also be indirectly confirmed with GATA-1 production inhibitory activity as an index. “Low” thrombocytopenic activity of a compound or a drug means that the action to decrease the platelet count is lower when the compound or the drug is administered to a human or a laboratory animal and the like, than when a compound used as a reference control is administered, and it is also possible to confirm whether or not the thrombocytopenic activity is low, by, for example, determining whether or not the GATA-1 production inhibitory activity is low by a method described above. As an example of the compound used as a reference control, Oxamflatin can be mentioned.

An “immunosuppressant” refers to an agent that induces immunosuppression; as an example of the immunosuppressant, an immunosuppressant having an activity to suppress IL-2 production can be mentioned. Therefore, the immunosuppressant can be obtained by, for example, determining whether or not it has IL-2 transcription suppressive activity using a method described above.

The present invention also relates to a method of selecting an IL-2 production inhibitor and/or an immunocyte proliferation inhibitor, each of which having low GATA-1 production inhibitory activity, or an immunosuppressant having low thrombocytopenic activity, which comprises measuring the HDAC4 and/or HDAC8 enzyme inhibitory activity of a test substance.

Specifically, the present invention relates to a method of selecting an IL-2 production inhibitor and/or an immunocyte proliferation inhibitor, each of which having low GATA-1 production inhibitory activity, or an immunosuppressant having low thrombocytopenic activity, which comprises (i) measuring the HDAC4 and/or HDAC8 enzyme inhibitory activity of a test substance, and (ii) measuring the HDAC enzyme inhibitory activity of the test substance (here, the HDAC enzyme inhibitory activity is one or more HDAC enzyme inhibitory activities selected from a group consisting of HDAC1, HDAC2, HDAC3, HDAC5, HDAC6 and HDAC7 enzyme inhibitory activities). In (ii) of this method, one or more HDAC enzyme inhibitory activities selected from a group consisting of HDAC1, HDAC2, HDAC3, HDAC5, HDAC6 and HDAC7 enzyme inhibitory activities are measured in the presence of the test substance; preferably all of HDAC1, HDAC2, HDAC3, HDAC5, HDAC6 and HDAC7 enzyme inhibitory activities are measured. This method may further comprises (iii) determining whether or not the test substance selectively inhibits HDAC4 and/or HDAC8 enzyme activity, on the basis of the results of (i) and (ii).

More specifically, the present invention relates to a method of selecting an IL-2 production inhibitor and/or an immunocyte proliferation inhibitor, each of which having low GATA-1 production inhibitory activity, or an immunosuppressant having low thrombocytopenic activity, wherein the IL-2 production inhibitor and/or the immunocyte proliferation inhibitor selectively inhibits HDAC4 and/or HDAC8 enzyme activity. In one embodiment of the present invention, the above-described method comprises (i) expressing the HDAC1, HDAC2, HDAC3, HDAC4, HDAC5, HDAC6, HDAC7 and HDAC8 genes to obtain respective enzyme solutions, then (ii) measuring HDAC enzyme activity for each of the enzyme solutions in the presence of a test substance, and (iii) selecting a test substance that selectively suppresses HDAC enzyme activity only when using an enzyme solution obtained by expressing the HDAC4 gene or the HDAC8 gene.

In another embodiment, the above-described method comprises (i) measuring HDAC enzyme activity for an enzyme solution partially purified from human cells, in the presence of a test substance, (ii) measuring HDAC enzyme activity for an enzyme solution obtained by expressing the HDAC4 gene and/or the HDAC8 gene, in the presence of the test substance, and (iii) selecting a test substance that selectively suppresses HDAC enzyme activity only when using an enzyme solution obtained by expressing the HDAC4 gene and/or the HDAC8 gene, by comparing the enzyme activities measured in (i) and (ii). The human cells used in (i) in this method are not subject to limitation, as long as they are of a human-derived cell line; preferably, a cell lines derived from the above-described immunocytes are used, and most preferably, Jurkat cells (ATCC accession number TIB-152) are used.

The above-described “test substance” is not subject to limitation, as long as it is a candidate substance that can be applied to an assay system according to the method of the present invention, and includes all compounds such as low-molecular organic compounds, low-molecular inorganic compounds, high-molecular compounds including proteins and nucleic acids, saccharides and the like, and also includes all of their mixtures, natural products, synthetic products, and extracts from animals, plants, fungi, algae and microorganisms. As examples of the “test substance”, HDAC inhibitors (see, for example, International Patent Publication Nos. WO02/085883, WO02/085400, WO02/076941, WO02/062773, WO02/46129, WO02/07722, WO02/06307, WO01/70675, WO01/38322, WO01/16106, WO00/71703, WO00/21979 and WO00/08048; Japanese Patent Publication Nos. 2001-348340 and HEI-11-302173; U.S. Patent Publications US 2002/0120099 and US 2002/0115826; U.S. Pat. Nos. 6,638,530, 6,541,661 and 6,399,568) can be used. The “test substance” may be a known compound or a newly discovered or synthesized compound.

An “HDAC inhibitor” refers to a compound that inhibits HDAC enzyme activity, that is, a compound having HDAC enzyme inhibitory activity.

“HDAC enzyme activity” refers to an activity to remove the acetyl group from the lysine residue of an acetylated protein, for example, a histone (i.e., deacetylation activity), and may be the deacetylation activity of a particular HDAC isozyme, or the deacetylation activity of one or two or more unspecified HDAC isozymes. On the other hand, “HDAC4 enzyme activity” refers to the deacetylation activity of HDAC4, and “HDAC8 enzyme activity” refers to the deacetylation activity of HDAC8. HDAC1 enzyme activity, HDAC2 enzyme activity, HDAC3 enzyme activity, HDAC4 enzyme activity, HDAC5 enzyme activity, HDAC6 enzyme activity, and HDAC7 enzyme activity are also defined similarly. HDAC enzyme activity can be measured by reacting an enzyme solution containing each HDAC enzyme (hereinafter abbreviated “enzyme solution” as required) and a substrate of the HDAC enzyme in the presence/absence of a test substance by a method known in the art.

“HDAC enzyme inhibitory activity” refers to an activity to suppress the deacetylation of the lysine residue of a protein, and may be an activity to specifically or non-specifically suppress the deacetylation by a particular HDAC isozyme. On the other hand, “HDAC4 enzyme inhibitory activity” refers to an activity to suppress the deacetylation by HDAC4, and “HDAC8 enzyme inhibitory activity” refers to an activity to suppress the deacetylation by HDAC8. HDAC1 enzyme inhibitory activity, HDAC2 enzyme inhibitory activity, HDAC3 enzyme inhibitory activity, HDAC4 enzyme inhibitory activity, HDAC5 enzyme inhibitory activity, HDAC6 enzyme inhibitory activity, and HDAC7 enzyme inhibitory activity are also defined similarly.

As the substrate used in the measurement of HDAC enzyme activity, a substrate known in the art can be used, and representatively, a [³H]acetyl-labeled histone, a fluorescently labeled acetylated peptide and the like can be used. For example, when a [³H]acetyl-labeled histone is used as a substrate, the [³H]acetyl-labeled histone can be obtained from Jurkat cells. Specifically, the following procedure is followed. 1×10⁸ Jurkat cells are incubated with 300 MBq [³H] sodium acetate in an RPMI-1640 medium supplemented with 10% FBS, penicillin (50 units/ml) and streptomycin (50 μg/ml), in the presence of 5 mM sodium butyrate, for 30 minutes (5% CO₂, 37° C.), the cells are recovered in a centrifugal tube (50 ml), collected via centrifugation (1000 rpm, 10 minutes) and once washed with phosphate-buffered saline. The washed cells are suspended in 15 ml of an ice-cooled lytic buffer (10 mM Tris-HCl, 50 mM sodium hydrogen sulfite, 1% Triton X-100, 10 mM MgCl₂, 8.6% sucrose, pH 6.5). After Dounce homogenization (30 strokes), nuclei are collected via centrifugation (1000 rpm, 10 minutes), washed with 15 ml of the lytic buffer three times, and then once washed with 15 ml of an ice-cooled washing buffer (10 mM Tris-HCl, 13 mM EDTA, pH 7.4). The pellet is suspended in 6 ml of ice-cooled water using a mixer, and 68 ml of H₂SO₄ is added to this suspension to obtain a concentration of 0.4N. After incubation at 4° C. for 1 hour, the suspension is centrifuged (15,000 rpm, 5 minutes), and the supernatant is mixed with 60 ml of acetone. Finally, overnight incubation at −20° C. is followed by recovery of the aggregate to yield a [³H]acetyl-labeled histone. For the method of obtaining a [³H]acetyl-labeled histone, see, for example, International Patent Publication No. WO00/08048, Yoshida, M. et al., J. Biol. Chem., 265: 17174-17179 (1990) and the like. The [³H]acetyl-labeled histone thus obtained may be used in solution in an appropriate buffer and immediately used as is, or if it is not used immediately, it may be stored under freezing or refrigeration in a solution state, or may be stored under freezing in a dry solid state.

To “express each gene” can be performed by incorporating the gene to an expression vector having a promoter sequence,. transferring the expression vector incorporating the gene to cells, and cultivating the cells, and the like. The promoter, expression vector and the like used differ variously depending on the host cells used for expression, and those skilled in the art having ordinary expertise can easily find the best combination in commercially available products. Alternatively, to “express each gene” can also be performed by transferring the gene to an expression vector having a promoter sequence, such as the SP6 phage or the T7 phage, and allowing the reaction in vitro by the in vitro transcription/translation method. Those skilled in the art having ordinary experimental knowledge can easily synthesize the transcription product and protein of each of the genes by this method.

As the method of transferring a gene used in the present invention to cells, a method of transformation known in the art, such as the calcium phosphate method, the liposome method, the lipofection method, the electroporation method and the like, can be used, which are not to be construed as limiting.

The cells to which a gene used in the present invention is transferred are not subject to limitation unless otherwise specified, as long as they are cells wherein a gene inserted in an expression vector is expressed, and mammalian cells including the above-described human cells, insect cells, yeast, Escherichia coli and the like can be used.

An “enzyme solution” refers to a fraction having HDAC enzyme activity extracted from a cell disruption solution or reaction solution having HDAC enzyme activity, wherein the HDAC enzyme may have been partially purified or purified to high purity, as long as the HDAC enzyme has been purified to the extent that enables a measurement of HDAC enzyme activity. A cell disruption solution having HDAC enzyme activity can be obtained from cells that are naturally expressing an HDAC enzyme, or cells wherein each HDAC gene is overexpressed. An enzyme solution can be obtained from cells that are naturally expressing an HDAC enzyme by, for example, cultivating the cells, and extracting a fraction having HDAC enzyme activity from a disruption solution of the cultured cells. Also, an enzyme solution can be obtained from cells allowed to overexpress each HDAC gene by, for example, inserting each HDAC gene in an expression vector, incorporating the expression vector to cells, cultivating the cells obtained, and extracting a fraction having HDAC enzyme activity from a disruption solution of the cultured cells. On the other hand, a reaction solution having HDAC enzyme activity can be prepared by, for example, synthesizing the transcription product and protein of each HDAC from each HDAC gene joined to an appropriate vector using the in vitro transcription/in vitro translation method.

“Partial purification” of an HDAC enzyme refers to purifying the HDAC enzyme to the extent that enables a measurement of HDAC enzyme activity, and can be conducted by a method known in the art, for example, a method described in International Patent Publication No. WO00/08048, Yoshida, M. et al., J. Biol. Chem. 265: p.17174-17179 (1990), and the like. Also, the partially purified HDAC enzyme can be purified to higher purity by another method of purification known in the art. For this partial purification, the following method of purification, for example, can be used. First, 5×10⁸ Jurkat cells are suspended in 40 ml of HDA buffer (composition: 15 mM potassium phosphate, 5% glycerol and 0.2 mM EDTA, pH 7.5). Subsequently, the suspension is homogenized, then nuclei are recovered via centrifugation (35,000×g, 10 minutes), and the nuclei obtained are homogenized in 20 ml of an HDA buffer supplemented with 1M (NH₄)₂SO₄. Subsequently, the homogenate is subjected to sonication and clarified by centrifugation (35,000×g, 10 minutes). Subsequently, deacetylase is precipitated by raising the (NH₄)₂SO₄ concentration to 3.5M. Subsequently, the precipitated protein is dissolved in 10 ml of HDA buffer, and this solution is dialyzed in 4 L of HDA buffer. After dialysis, the sample solution is loaded to a DEAE-cellulose (Whatman DE52) column (25×85 mm), previously equilibrated with HDA buffer, and eluted with 300 ml of NaCl solution on a linear gradient (0 to 0.6M). As a result, HDAC enzyme activity appears as a single peak in the fraction eluted at 0.3 to 0.4M NaCl.

HDAC enzyme activity is measured by a method known in the art. For example, when a [³H]acetyl-labeled histone is used as a substrate, 10 μl of the [³H]acetyl-labeled histone is added to 90 μl of enzyme solution, and this mixture is incubated at 25° C. for 30 minutes. The reaction is stopped by the addition of 10 μl of hydrochloric acid, and the released [³H] acetic acid is extracted with 1 ml of ethyl acetate. Finally, 0.9 ml of the solvent layer is transferred to a toluene scintillation solution, and the radioactivity of this solution is measured, thereby HDAC enzyme activity can be determined. When the HDAC enzyme activity of each enzyme solution is measured in the presence of a test substance, the test substance may be added around the time of preparation of a mixture of 10 μl of the [³H]acetyl-labeled histone and 90 μl of the enzyme solution, and the reaction may be initiated. For details about the measurement of HDAC enzyme activity, see, for example, International Patent Publication No. WO00/08048; Yoshida, M. et al., J. Biol. Chem., 265: 17174-17179 (1990) and the like, when a [³H]acetyl-labeled histone is used as a substrate, and see, for example, International Patent Publication No. WO01/040506 and the like, when a fluorescently labeled acetylated peptide is used as a substrate.

In the above-described method, a test substance that selectively suppresses HDAC enzyme activity only when using an enzyme solution obtained by expressing the HDAC4 gene or the HDAC8 gene is preferably selected, and a test substance that suppresses the HDAC enzyme activity of HDAC4 and/or HDAC8 more than suppresses the HDAC enzyme activity of HDAC1-3 and HDAC5-7 may be selected. More preferably, a test substance that does not suppress or substantially does not suppress the HDAC enzyme activity of HDAC1-3 and HDAC5-7, and that suppresses the HDAC enzyme activity of HDAC4 and/or HDAC8, is selected.

The present invention also relates to a method of selecting an IL-2 production inhibitor and/or an immunocyte proliferation inhibitor, each of which having low GATA-1 production inhibitory activity, or an immunosuppressant having low thrombocytopenic activity, which comprises selecting an inhibitor that specifically inhibits the formation of a complex of HDAC4 and N-COR from among test HDAC inhibitors. For example, this method comprises (i) expressing the HDAC4 gene and the N-CoR gene to obtain an enzyme solution, then (ii) allowing the enzyme solution to coexist with a test HDAC inhibitor, and (iii) determining whether or not a complex of HDAC4 and N-COR is formed in the mixture of enzyme solution and test HDAC inhibitor obtained in (ii) In this method, an enzyme solution obtained by expressing the HDAC3 gene, in addition to the HDAC4 gene and the N-COR gene, may also be used. In this case, whether or not a higher complex is formed between HDAC4 and N-CoR and HDAC3 in the presence of a test HDAC inhibitor is determined. In (iii) in the above-described method, as examples of the method of determining whether or not a complex is formed, a method known in the art such as an analytical method using surface plasmon resonance, an analytical method using an antibody (for example, Western blotting) and the like can be mentioned.

As the labeled substance used to label the secondary antibody in an analytical method using an antibody, for example, Western blotting, an enzyme such is as horseradish peroxidase and alkaline phosphatase, a fluorescent substance such as fluorescein isocyanate and rhodamine, a radioactive substance such as ³²p or ¹²⁵I, a chemical luminescent substance and the like can be mentioned.

To select an IL-2 production inhibitor and/or an immunocyte proliferation inhibitor, each of which having low GATA-1 production inhibitory activity, or an immunosuppressant having low thrombocytopenic activity, that specifically inhibits the formation of a complex of HDAC4 and N-CoR, a method based on a two-hybrid system known in the art can also be used. For example, when a two-hybrid system is used for this purpose, a test substance that specifically inhibits the formation of a complex of an HDAC4 fusion protein comprising at least a portion of the HDAC4 protein and an N-CoR fusion protein comprising at least a portion of the N-COR protein is selected by (i) expressing a gene that encodes the HDAC4 fusion protein and a gene that encodes the N-COR fusion protein in cells, and (ii) determining whether or not a test substance (preferably a test HDAC inhibitor) inhibits the formation of a complex of the HDAC4 fusion protein and the N-CoR fusion protein.

The HDAC4 fusion protein may be any one comprising a region that interacts with N-CoR in the HDAC4 protein, and may also comprise the entire HDAC4 protein. Likewise, the N-COR fusion protein may be any one comprising a region that interacts with HDAC4 in the N-CoR protein (for example, RD3 described below), and may also comprise the entire N-CoR protein. Each of the HDAC4 fusion protein and the N-COR fusion protein further comprises, in addition to a portion or the entire region of the HDAC4 protein and the N-COR protein, a DNA-binding domain (for example, GAL4 DNA-binding domain and the like) or a transcription activation domain (for example, VP16 activation domain and the like) derived from transcription regulatory factor. However, if the HDAC4 fusion protein comprises a DNA-binding domain, the N-CoR fusion protein need to comprise a transcription activation domain; if the HDAC4 fusion protein comprises a transcription activation domain, the N-COR fusion protein need to comprise a DNA-binding domain.

Whether or not a complex is formed between the two fusion proteins can be confirmed by the expression level of a gene located downstream of the promoter to which the DNA-binding region in a fusion protein binds. Accordingly, when a complex is formed between the fusion proteins, the expression level of the mRNA or protein encoded by the gene, and the expression level of a reporter gene comprising a promoter located upstream of the gene, increase, whereas when a complex is not formed between the fusion proteins, the expression level of the mRNA or protein encoded by the gene, and the expression level of the above-described reporter gene comprising a promoter remain unchanged.

Whether or not a test substance (preferably a test HDAC inhibitor) inhibits the formation of a complex of an HDAC4 fusion protein and an N-CoR fusion protein can be determined by determining whether or not the expression level of a gene located downstream of the promoter to which the DNA-binding region in a fusion protein binds is decreased in the presence of the test substance. Accordingly, (a) the expression level of the mRNA or protein encoded by the gene or the expression level of a reporter gene comprising a promoter located upstream of the gene is measured in the absence of a test substance, (b) the expression level of the mRNA, protein or reporter gene is measured in the presence of the test substance, and (c) comparing the expression level measured in (a) and the expression level measured in (b); if the expression level measured in (b) is lower than the expression level measured in (a), the test substance is selected as a substance that inhibits the formation of a complex of an HDAC4 fusion protein and an N-CoR fusion protein. It is preferable, from the viewpoint of assay simplicity, sensitivity and the like, that whether or not the test substance inhibits the formation of a complex of an HDAC4 fusion protein and an N-COR fusion protein be evaluated by measuring the expression level of a reporter gene.

The present invention also relates to a method of selecting a compound having low GATA-1 production inhibitory activity, and having IL-2 production inhibitory activity and/or immunocyte proliferation inhibitory activity, or a compound having low thrombocytopenic activity and having immunosuppressive activity, which comprises selecting a compound that suppresses the expression level of HDAC4 and/or HDAC8 from among test HDAC inhibitors. This method comprises (i) allowing cells that express HDAC4 and/or HDAC8 to coexist with a test HDAC inhibitor, and (ii) measuring the HDAC4 and/or HDAC8 expression level in the cells. Specifically, this method can be conducted in the same manner as the method described below of measuring the expression level of GATA-1 mRNA, GATA-1 protein or GATA-1 reporter gene.

The present invention also relates to a method of selecting a compound having low GATA-1 production inhibitory activity and having IL-2 production inhibitory activity, or a compound having low thrombocytopenic activity and having immunosuppressive activity, wherein an HDAC inhibitor that selectively inhibits the binding of HDAC4 and/or HDAC8 and an HDAC4- and/or HDAC8-specific ligand is selected from among test HDAC inhibitors. This method comprises (i) expressing the HDAC4 and/or the HDAC8 gene to obtain an enzyme solution, and then (ii) measuring the binding activity of HDAC4 and/or HDAC8 enzyme and the HDAC4- and/or HDAC8-specific ligand for the enzyme solution in the presence of an HDAC4- and/or HDAC8-specific ligand and a test HDAC inhibitor.

An “HDAC4- and/or HDAC8-specific ligand” refers to a substance showing stronger binding activity to HDAC4 and/or HDAC8 than to HDAC1-3 and HDAC5-7, out of substances that specifically bind to HDAC4 and/or HDAC8. These substances include, but are not limited to, substances that promote the activity of each enzyme, those that inhibit the same, and the like. As “the HDAC4- and/or HDAC8-specific ligand”, a low-molecular substance that shows weak binding activity to HDAC1-3 and HDAC5-7 or is substantially free from binding activity, and that shows strong binding activity to HDAC4 and/or HDAC8, is preferred.

In step (ii) in the above-described method, an “HDAC4- and/or HDAC8-specific ligand” is preferably used, but an “HDAC4- and/or HDAC8-nonspecific ligand” can also be used. An “HDAC4- and/or HDAC8-nonspecific ligand” refers to a substance that shows stronger binding activity to HDAC1-3 and HDAC5-7 than to HDAC4 and/or HDAC8, or a substance that shows nearly equivalent binding activity to HDAC4 and/or HDAC8 and to HDACl-3 and HDAC5-7. In the present invention, an “HDAC4- and/or HDAC8-specific ligand” and an “HDAC4- and/or HDAC8-nonspecific ligand” are also integrally referred to as an “HDAC4 and/or HDAC8 ligand”.

In step (ii) in the above-described method, the binding activity of HDAC4 and/or HDAC8 enzyme and an HDAC4- and/or HDAC8-specific ligand is measured in the presence of a test HDAC inhibitor. Binding activity can be measured using, for example, a method known in the art, such as binding assay or mass analysis. By the above-described method, a substance that competitively inhibits an HDAC4- and/or HDAC8-specific ligand can be obtained.

In the present invention, human-derived HDAC4 is preferably used as HDAC4. Specifically, HDAC4 refers to, for example, a protein that comprises the amino acid sequence shown by SEQ ID NO:2 or an analogue thereof that has HDAC enzyme activity (for example, protein encoded by the base sequence of GenBank accession number AF132607). HDAC4 analogues include a variant having one or two or more amino acids substituted, deleted or added in-the amino acid sequence shown by SEQ ID NO:2, a sugar chain added form and the like. As the HDAC4 analogue, a protein that comprises an amino acid sequence having a homology of 70% or more, preferably 80% or more, more preferably 90% or more, still more preferably 95% or more, to the amino acid sequence shown by SEQ ID NO:2, can also be used. The degree of amino acid sequence homology can be determined by, for example, using BLAST in the default settings between the amino acid sequence of interest and a reference amino acid sequence in NCBI (The National Center for Biotechnology Information). For details about HDAC4, see, for example, International Patent Publication No. WO00/10583.

Likewise, in the present invention, human-derived HDAC8 is preferably used as HDAC8. Specifically, HDAC8 refers to, for example, a protein that comprises the amino acid sequence shown by SEQ ID NO:8, or an analogue thereof that has HDAC enzyme activity (for example, protein encoded by the base sequence of GenBank accession number HSA277724). HDAC8 analogues include a variant having one or two or more amino acids substituted, deleted or added in the amino acid sequence shown by SEQ ID NO:8, a sugar chain added form and the like. As the HDAC8 analogue, a protein that comprises an amino acid sequence having a homology of 70% or more, preferably 80% or more, more preferably 90% or more, still more preferably 95% or more, to the amino acid sequence shown by SEQ ID NO:8 can also be used. For details about HDAC8, see, for example, Joseph J. BUGGY et al., Biochemical Journal 350: 199-205 (2000).

In the present invention, human-derived N-COR is preferably used as N-COR. Specifically, N-CoR refers to, for example, a protein that comprises the amino acid sequence shown by SEQ ID NO:22, or an analogue thereof that is capable of forming a complex with HDAC4 or HDAC3 (for example, protein encoded by the base sequence of GenBank accession number AF044209). N-COR analogues include a variant having one or two or more amino acids substituted, deleted or added in the amino acid sequence shown by SEQ ID NO:22, a sugar chain added form and the like. As the N-CoR analogue, a protein that comprises an amino acid sequence having a homology of 70% or more, preferably 80% or more, more preferably 90% or more, still more preferably 95% or more, to the amino acid sequence shown by SEQ ID NO:22 can also be used. In the present invention, N-CoR may be any one, as long as it is capable of forming a complex with HDAC4 (both HDAC4 and HDAC3 as required). As such N-CoR, the N-COR variants disclosed in, for example, Matthew G. Guenther et al., Molecular and Cellular Biology, 21(18): 6091-6101 (2001) and elsewhere, and the like can also be used, in addition to those mentioned above.

In the present invention, human-derived HDAC3 is preferably used as HDAC3. Specifically, HDAC3 refers to, for example, a protein that comprises the amino acid sequence shown by SEQ ID NO:16, or an analogue thereof that has HDAC enzyme activity or is capable of binding to HDAC4 (HDAC4—N-CoR complex as required) (for example, protein encoded by the base sequence of GenBank accession number U66914). HDAC3 analogues include a variant having one or two or more amino acids substituted, deleted or added in the amino acid sequence shown by SEQ ID NO:16, a sugar chain added form and the like. As the HDAC3 analogue, a protein that comprises an amino acid sequence having a homology of 70% or more, preferably 80% or more, more preferably 90% or more, still more preferably 95% or more, to the amino acid sequence shown by SEQ ID NO:16 can also be used. In the analysis of a complex of HDAC3 and N-CoR (a higher complex of HDAC3 and HDAC4 - N-CoR complex as required), HDAC3 need not always have HDAC enzyme activity, as long as it retains the capability of binding to HDAC4 (HDAC4—N-CoR complex as required). For details about HDAC3, see, for example, Molecular Cell, 9: 45-57 (January 2002), Matthew G. Guenther et al., Molecular and Cellular Biology, 21(18): 6091-6101 (2001) and the like.

Furthermore, in the present invention, human-derived HDAC1-2 and HDAC5-7 are preferably used as HDAC1-2 and HDAC5-7. Specifically, each of HDAC1-2 and HDAC5-7 is a protein encoded by the base sequence of GenBank accession number D50405 (HDAC1), the base sequence of GenBank accession number HSU31814 (HDAC2), the base sequence of GenBank accession number AF132608 (HDAC5), the base sequence of GenBank accession number AF132609 (HDAC6), or the base sequence of GenBank accession number AF239243 (HDAC7), or an analogue thereof that has HDAC enzyme activity. HDAC1-2 and HDAC5-7 analogues include a variant having one or two or more amino acids substituted, deleted or added in the amino acid sequence corresponding to each of the above-described HDAC1-2, HDAC5-7, a sugar chain added form and the like. As the HDAC1-2 and HDAC5-7 analogues, a protein that comprises an amino acid sequence having a homology of 70% or more, preferably 80% or more, more preferably 90% or more, still more preferably 95% or more, to the amino acid sequence corresponding to each of the above-described HDAC1-2 and HDAC5-7, can also be used

Furthermore, the present invention relates to a method of selecting a selective HDAC4 and/or HDAC8 inhibitor having low thrombocytopenic activity by further confirming the degree of GATA-1 production inhibitory activity of an immunosuppressant and/or an immunocyte proliferation inhibitor, each of which having low thrombocytopenic activity selected as described above. Although the degree of GATA-1 production inhibitory activity can be evaluated by, for example, measuring the expression level of GATA-1 mRNA, GATA-1 protein or GATA-1 reporter gene in a cell such as cell line or primary culture cells that are naturally expressing GATA-1, in the presence and/or absence of an immunosuppressant having low thrombocytopenic activity, it is preferable, from the viewpoint of assay simplicity, sensitivity and the like, that the expression level of the GATA-1 reporter gene be measured.

In the above-described method, as the test cells used to measure GATA-1 production inhibitory activity, a cell line, primary culture cells and the like that are naturally expressing GATA-1 can be used. Whether or not a particular cell line or primary culture cells are naturally expressing GATA-1 can be determined by confirming the expression of GATA-1 mRNA or protein in these cells by a method known in the art. Also, as the test cells used in the above-described method, cells that substantially reflect the state of megakaryocytic lineage cell are also preferred. The cell line that substantially reflects the state of megakaryocytic lineage cell is not subject to limitation, as long as it is derived from megakaryocytic lineage cell; for example, human-derived HEL cells (accession number JCRB0062, Japanese Collection of Research Bioresources) can be used.

When the GATA-1 production inhibitory activity of an HDAC4 and/or HDAC8 inhibitor is evaluated by the expression level of GATA-1 mRNA or GATA-1 protein, test cells can be allowed to coexist with the HDAC4 and/or HDAC8 inhibitor by adding the HDAC4 and/or HDAC8 inhibitor to a cell culture, and cultivating the cells.

When the GATA-1 production inhibitory activity of an HDAC4 and/or HDAC8 inhibitor is evaluated by a reporter assay using a GATA-1 reporter gene, test cells can be allowed to coexist with the HDAC4 and/or HDAC8 inhibitor by adding the HDAC4 and/or HDAC8 inhibitor to a cell culture, around the time of transferring the GATA-1 reporter gene to the cells by a transfection method such as electroporation. Preferably, the GATA-1 reporter gene is transferred, then the HDAC4 and/or HDAC8 inhibitor is added to the culture, and further cultivate the cells.

The expression level of GATA-1 mRNA can be confirmed by, for example, preparing each total RNA from test cells, and comparing the expression level of GATA-1 mRNA by quantitative RT-PCR and the like known in the art, such as competitive RT-PCR or real-time RT-PCR and the like.

The expression level of GATA-1 protein can be measured by, for example, preparing an extract from cells allowed to exist in the presence and absence of an HDAC4 and/or HDAC8 inhibitor, and using an anti-GATA-1 antibody and a labeled secondary antibody. As the method for this measurement, various methods in use for general immunochemical assays, such as radioisotope immunoassay (RIA), ELISA [Engvall, E., Methods in Enzymol., 70, 419-439 (1980)], fluorescent antibody method, plaque method, spot method, agglutination method, and Ouchterlony method (“Hybridoma Method and Monoclonal Antibodies”, published by R&D Planning, pp. 30-53, March 5, 1982) can be used, and these methods can be easily performed by those skilled in the art. Although one of the above-described methods can be appropriately selected from various viewpoints, ELISA is preferred from the viewpoint of sensitivity, ease of operation and the like. As the labeling substance used to label the above-described secondary antibody, the same as those mentioned above can be used.

The expression level of a GATA-1 reporter gene can be evaluated by a reporter assay using the GATA-1 reporter gene. The “GATA-1 reporter gene” refers to a DNA construct prepared by artificially joining the transcription control region of the GATA-1 gene and a reporter gene. As the transcription control region of the GATA-1 gene, a DNA fragment comprising both the IE promoter region of the human GATA-1 gene (GenBank accession number: AF196971) and the HSI region (highly-sensitive-to-DNase I region) located at about 3.5 kb apart upstream thereof is used. Regarding the scope of transcription control region essential to the objects of the present invention, the region is desirably one that substantially reflects the mode of transcription control of the natural GATA-1 gene in megakaryocyte lineage cells, rather than simply retains transcriptional activity.

For promoters of the GATA-1 gene, at least two kinds, i.e., the IE promoter and the IT promoter, are known to exist. In relation to the thrombocytopenic activity of HDAC inhibitors, because the function of the IE promoter, which has been shown to be closely associated with transcription control in megakaryocyte lineage cells, is stipulated to be important, its control region is preferably used.

For the transcription control by the IE promoter, both a sequence located within about 0.7 kb just upstream of the transcription initiation site and the HSI region located further upstream thereof (highly-sensitive-to-DNase I region) are important. On the 5′-side of the HSI region, two CACC sites are present, and apart therefrom by about 50 base pairs, a GATA binding site and the E-box motif are adjacent to each other by a distance of about 10 base pairs. It is known that if a mutation that destroys the GATA-binding site is introduced, erythrocyte- or megakaryocyte-specific expression is no longer observed (P. Vyas et al., Development 126; 2799-2811 (1999)). Also, further on the 3′-side of this GATA-E-box motif, the CTGTGGCCACAG sequence (SEQ ID NO:86), which is a characteristic palindrome sequence, and a GC-rich region are present. In the GC-rich region, the GGAA sequence, which is seen in the binding site of the transcription factor ETS, is present. Even if the CACC site on the 5′-side is deleted from the GATA-1 HSI region comprising these sequences, erythrocyte- or megakaryocyte-specific expression is seen, but if the GATA-E-box motif is also deleted, this expression disappears. Regarding the 3′-side, it has been reported that the GATA-1 HSI region can function as an enhancer, provided that up to about 250 base pairs downstream of the E-box are included. Therefore, provided that all the essential sequence sites described above are contained, the sequence may actually be of any length; for example, a sequence prepared by artificially joining a region comprising 819 base pairs in the vicinity of the transcription initiation site by the IE promoter (−789 to +30: transcription initiation site taken as +1) of the human the GATA-1 gene, and a region comprising 637 base pairs (−3769 to −3133) as the HIS region upstream of the transcription initiation site, can be used.

For the essential sequence sites such as the GATA-E-box motif, it is possible to prepare an artificial DNA construct wherein the essential sequences are multiplied tandem in a plurality of sets by an ordinary technique of recombinant DNA experiments. In some cases, the transcription induction activity of the transcription control region can be enhanced by using such an artificial construct in place of the natural sequence.

Note that a “reporter gene” refers to a gene that comprises a structural gene region that encodes a specific protein serving as an index of gene expression and a downstream untranslated region (3′-non-coding region), and that lacks all the transcription control region originally present on the upstream side of the structural gene. The structural gene region that encodes a specific protein serving as an index of gene expression may be any one, as long as the gene expression from the transcription control region in an exogenous gene fragment can easily be measured from the function of the index protein encoded by the reporter gene by artificially joining the structural gene region and the exogenous gene fragment, and transferring the structural gene region and exogenous gene fragment to cells. More preferably, the index protein is desirably one that stably occurs in ordinary mammalian cells, and that has no endogenous protein having similar activity in the cells, or has such an endogenous protein that can be measured with easy distinguishment. More preferably, the mRNA that encodes the index protein is desirably one that stably occurs in cells. Also, the substrate for the index protein is desirably one that need not be newly added, or can easily be incorporated in cells in a sufficient amount if need to be added, in measuring the activity. The assay system composed thereof desirably has both a broad range of linearity and high sensitivity. It is possible to use the firefly-derived luciferase gene, the Renilla-derived luciferase gene, the alkaline phosphatase gene, the β-galactosidase gene, the green fluorescent protein (GFP) gene, the enhanced green fluorescent protein (EGFP) gene, the β-glucuronidase gene, the chloramphenicol acetyltransferase (CAT) gene, the horseradish peroxidase (HRP) gene and the like. More preferably, the firefly-derived luciferase gene is used. For the firefly-derived luciferase gene, the detection sensitivity of the measurement system can be increased by using an improved type of the luciferase gene (luc+). For the untranslated region downstream of the structural gene, a sequence derived from the SV40 virus genome late gene, for example, can be used.

Each of the above-described GATA-1 reporter genes is joined to a cloning vector and amplified with Escherichia coli as a host. The cloning vector used is not subject to limitation, as long as it has a replication origin amplifiable in Escherichia coli and a selection marker; for example, using pGL3-Basic (Promega Corporation), which encodes an ampicillin resistance marker and an improved type of the luciferase structural gene (luc+) and the like, the above-described DNA construct can easily be prepared. Subsequently, the amplified vector is transiently transferred to cells and reporter activity is measured.

The present invention also relates to a kit for the selection of an immunosuppressant having low thrombocytopenic activity, or an IL-2 production inhibitor and/or an immunocyte proliferation inhibitor, each of which having low GATA-1 production inhibitory activity and the like, which includes all or part of the above-described components necessary to perform the above-described method. As an example of the assay kit for the selection of an immunosuppressant having low thrombocytopenic activity comprising a selective HDAC4 and/or HDAC8 inhibitor, a kit including (i) enzyme solutions obtained by expressing the HDAC1, HDAC2, HDAC3, HDAC5, HDAC6 and HDAC7 genes, respectively, or (ii) an enzyme solution obtained by expressing each of the HDAC4 and/or HDAC8 gene can be mentioned. Furthermore, the above-described kit may comprise other components, an instruction manual and the like.

The present invention encompasses a compound such as an immunosuppressant having low thrombocytopenic activity, or an IL-2 production inhibitor and/or an immunocyte proliferation inhibitor, each of which having low GATA-1 production inhibitory activity, obtained by the above-described method, or using the above-described kit.

Furthermore, the compound obtained by the above-described method, or using the above-described kit, is useful as an immunosuppressant having low thrombocytopenic activity, an IL-2 production inhibitor having low GATA-1 production inhibitory activity, and a selective HDAC4 and/or HDAC8 inhibitor having low thrombocytopenic activity, in the treatment or prophylaxis of diseases such as inflammatory diseases, diabetes, diabetic complications, homozygous thalassemia, fibrosis, liver cirrhosis, acute promyelocytic leukemia, protozoan infections, graft rejection during the transplantation of organs or tissues such as heart, kidney, liver, bone marrow, skin, cornea, lung, pancreas, small intestine, hand and foot, muscle, nerve, intervertebral disc, trachea, myeloblasts, and cartilage, graft-versus-host reactions in bone marrow transplantation, autoimmune diseases, cancers and the like. The present invention relates to a therapeutic and/or prophylactic pharmaceutical for the above-described diseases, which comprises such a compound as an active ingredient, and a therapeutic and/or prophylactic method for the above-described diseases, which comprises administering an effective amount of such a compound.

When the compound obtained by the above-described method, or using the above-described kit, is used as a pharmaceutical, it can be used as a pharmaceutical as is, and can also be used after being prepared as a formulation by a publicly known method of pharmaceutical making. For example, it can be used in the form of a pharmaceutical formulation which is appropriately combined with a publicly known pharmacologically acceptable carrier or medium, specifically distilled water, saline, vegetable oil, emulsifier, suspending agent and the like, for example, a solid, semi-solid or liquid formulation (for example, tablets, pills, troches, capsules, suppositories, creams, ointments, aerosols, powders, solutions, emulsions, suspensions and the like).

Regarding administration to patients, the pharmaceutical formulation of the present invention is suitable for nasal, ophthalmic, external (topical), rectal, pulmonary (nasal or intraoral injection), oral or non-oral (including subcutaneous, intravenous and intramuscular) administration or inhalation. Administration of an injection can be conducted by a publicly known method, for example, intra-arterial injection, intravenous injection, subcutaneous injection and the like. Dosage is variable depending on body weight, age and symptoms of patient, and the method of administration used and the like, and those skilled in the art can appropriately select the appropriate dosage.

The present invention also relates to a variant that lacks the function of normal HDAC4 enzyme (i.e., an HDAC4 dominant negative variant). As an example of the HDAC4 dominant negative variant, an HDAC4 variant that has (consists of) the amino acid sequence shown by SEQ ID NO:4 or SEQ ID NO:6 can be mentioned. Furthermore, the present invention also encompasses a variant that has one or more amino acids deleted, substituted or added in the amino acid sequence shown by SEQ ID NO:4 or SEQ ID NO:6, and that lacks the function of normal HDAC4 enzyme.

Furthermore, the present invention relates to a DNA that has (consists of) the base sequence that encodes the above-described HDAC4 dominant negative variant. More specifically, the present invention relates to a DNA that has (consists of) the base sequence shown by SEQ ID NO:3 or SEQ ID NO:5. The present invention also encompasses a DNA that has the base sequence that encodes a variant that has one or more amino acids deleted, substituted or added in the amino acid sequence shown by SEQ ID NO:4 or SEQ ID NO:6, and that lacks the function of normal HDAC4 enzyme.

The present invention also relates to a variant that lacks the function of normal HDAC1 enzyme (i.e., an HDAC1 dominant. negative variant). As an example of the HDAC1 dominant negative variant, an HDAC1 variant that has (consists of) the amino acid sequence shown by SEQ ID NO:12 or SEQ ID NO:14 can be mentioned. Furthermore, the present invention also encompasses a variant that has one or more amino acids deleted, substituted or added in the amino acid sequence shown by SEQ ID NO:12 or SEQ ID NO:14, and that lacks the function of normal HDAC1 enzyme.

Furthermore, the present invention relates to a DNA that has (consists of) the base sequence that encodes the above-described HDAC1 dominant negative variant. More specifically, the present invention relates to a DNA that has (consists of) the base sequence shown by SEQ ID NO:11 or SEQ ID NO:13. The present invention also encompasses a DNA that has the base sequence that encodes a variant that has one or more amino acids deleted, substituted or added in the amino acid sequence shown by SEQ ID NO:12 or SEQ ID NO:14, and that lacks the function of normal HDAC1 enzyme.

The present invention also relates to a variant that lacks the function of normal HDAC3 enzyme (i.e., an HDAC3 dominant negative variant). As an example of the HDAC3 dominant negative variant, an HDAC3 variant that has (consists of) the amino acid sequence shown by SEQ ID NO:18 or SEQ ID NO:20, can be mentioned. Furthermore, the present invention also encompasses a variant that has one or more amino acids deleted, substituted or added in the amino acid sequence shown by SEQ ID NO:18 or SEQ ID NO:20, and that lacks the function of normal HDAC3 enzyme.

Furthermore, the present invention relates to a DNA that has (consists of) the base sequence that encodes the above-described HDAC3 dominant negative variant. More specifically, the present invention relates to a DNA that has (consists of) the base sequence shown by SEQ ID NO:17 or SEQ ID NO:19. The present invention also encompasses a DNA that has the base sequence that encodes a variant that has one or more amino acids deleted, substituted or added in the amino acid sequence shown by SEQ ID NO:18 or SEQ ID NO:20, and that lacks the function of normal HDAC3 enzyme.

HDAC1, HDAC3 and HDAC4 dominant negative variants, enzyme solutions containing these variants, and DNAs that encode these variants can, for example, be used as negative controls in the above-described screening. The kit for performing the screening method of the present invention can also include these HDAC1, HDAC3 and HDAC4 dominant negative variants, enzyme solutions containing these variants, or DNAs that encode these variants.

### EXAMPLES

The present invention is hereinafter described in more detail by means of the following Examples, which examples, however, are not to be construed as limiting the scope of the present invention.

### Example 1

### Construction of Reporter Gene Plasmids for IL-2 Reporter Gene Assay

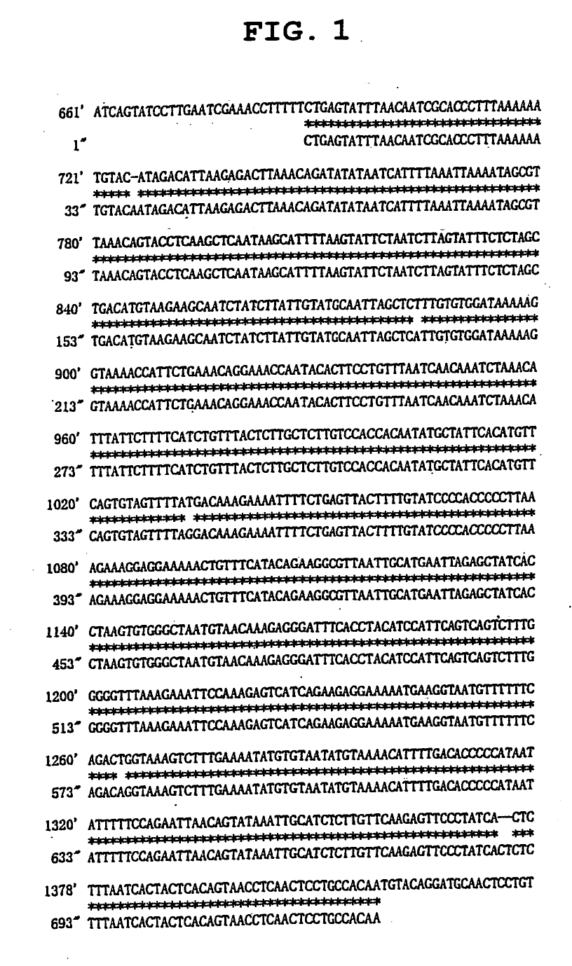

A 728-basepair fragment corresponding to the −674 to +54 region in the vicinity of the transcription initiation site of the human IL-2 gene was obtained by the PCR method with a genomic DNA isolated from Jurkat cells derived from human T cell (ATCC, TIB-152) as a template. PCR was performed using the primers shown by SEQ ID NO:27 and SEQ ID NO:28 below. These primers were designed on the basis of the human IL-2 gene sequence listed as Locus code: HSIL05 in the gene database GenBank. Additionally, a restriction endonuclease recognition site was added to the end of each primer to insert to a vector for reporter gene assay. The 728-basepair fragment obtained has an NheI recognition site upstream of the promoter, and a HindIII recognition site downstream of the promoter. The amplified 728-basepair fragment was inserted to the cloning vector pCR4 (manufactured by Invitrogen). Subsequently, the base sequence of the inserted region in the plasmid obtained (SEQ ID NO:23) was confirmed. As a result, the base sequence of the inserted region (SEQ ID NO:23) was confirmed as having base substitution at three sites, insertion of two bases at one site, and insertion of one base at one site, as shown in FIG. 1, compared to the promoter sequence of IL-2 listed as GenBank Locus code: HSIL05 (SEQ ID NO:29). Subsequently, the plasmid obtained was cleaved at the NheI recognition site and the HindIII recognition site. The fragment obtained was inserted to the NheI/HindIII site of pGL3 basic, a vector for reporter gene assay comprising the firefly luciferase gene (manufactured by Promega Company). Thus, the plasmid pGL3 IL2 Pro was obtained, which has a 731-basepair sequence (SEQ ID NO:23) corresponding to the −675 to +56 region in the vicinity of the transcription initiation site of the human IL-2 gene upstream of the firefly luciferase gene.

| | |
|---|---|
| TCGCTAGCCTGAGTATTTAACAATCGCACCCT | (SEQ ID NO:27) |
| CGAAGCTTGTGGCAGGAGTTGAGGTTACTG | (SEQ ID NO:28) |

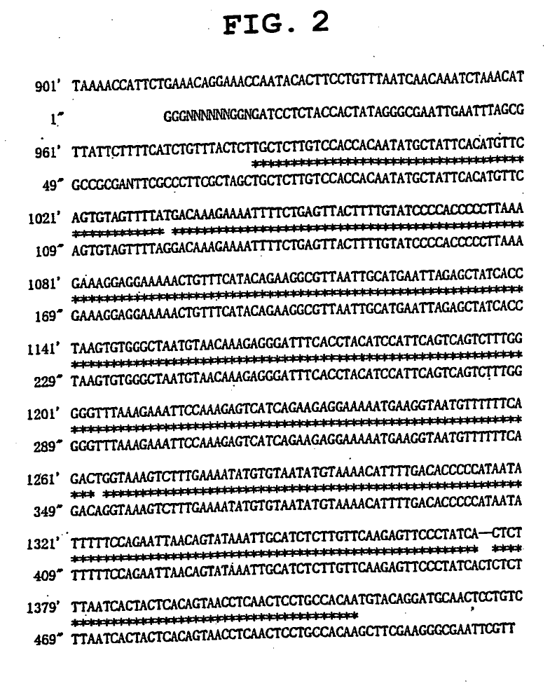

Subsequently, a 435-basepair fragment corresponding to the −379 to +56 region in the vicinity of the transcription initiation site of the human IL-2 gene was obtained by the PCR method with pGL3 IL2 Pro as a template. PCR was performed using the primer shown by SEQ ID NO:30 below and the primer shown by SEQ ID NO:28 above. The SEQ ID NO:30 primer was designed on the basis of the human IL-2 gene sequence inserted in pGL3 IL2 Pro. Additionally, a restriction endonuclease recognition site was added to the end of each primer to insert to a vector for reporter gene assay. The 435-basepair fragment obtained has an NheI recognition site upstream of the promoter, and a HindIII recognition site downstream of the promoter. The amplified 435-basepair fragment was inserted to the cloning vector pCR4. Subsequently, the base sequence of the inserted region in the plasmid obtained (SEQ ID NO:24) was confirmed. As a result, the base sequence of the inserted region (SEQ ID NO:24) was confirmed as having base substitution at two sites and insertion of two bases at one site as shown in FIG. 2 compared to the promoter sequence of IL-2 listed as GenBank Locus code: HSIL05 (SEQ ID NO:31). Subsequently, the plasmid obtained was cleaved at the NheI recognition site and the HindIII recognition site. The fragment obtained was inserted to the NheI/HindIII site of pGL3, a vector for reporter gene assay comprising the firefly luciferase gene. Thus, the plasmid pGL3 IL2 Pro43 was obtained, which has a 435-basepair promoter sequence corresponding to the −379 to +56 region in the vicinity of the transcription initiation site of the human IL-2 gene upstream of the firefly luciferase gene.

| | |
|---|---|
| CGCTAGCTGCTCTTGTCCACCACAATATGC | (SEQ ID NO:30) |

### Example 2

### Construction of Reporter Gene Plasmids for GATA-1 Reporter Gene Assay

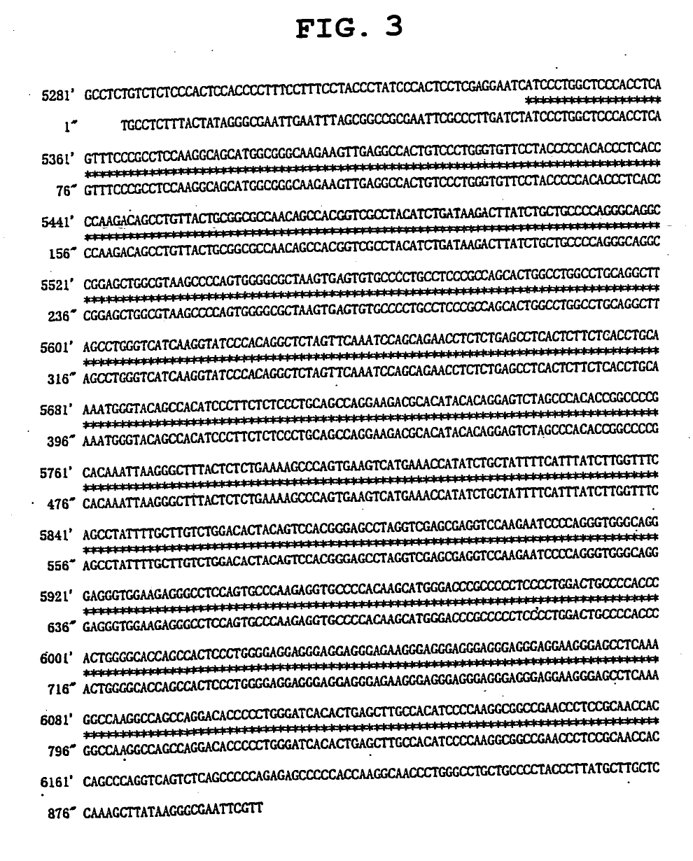

An 821-basepair fragment corresponding to the −789 to +32 region in the vicinity of the transcription initiation site of the human GATA-1 gene was obtained by the PCR method with human genomic DNA as a template. PCR was performed using the primers shown by SEQ ID NO:32 and SEQ ID NO:33 below. These primers were designed on the basis of the human GATA-1 gene sequence listed as gene database GenBank accession number AF196971. Additionally, a restriction endonuclease recognition site was added to the end of each primer to insert to a vector for reporter gene assay. The 821-basepair fragment obtained has a BglII recognition site upstream of the promoter, and a HindIII recognition site downstream of the promoter. The amplified 821-basepair fragment was inserted to the cloning vector pCR4. Subsequently, the base sequence of the inserted region in the plasmid obtained (SEQ ID NO:25) was confirmed. As a result, the base sequence of the inserted region (SEQ ID NO:25) was consistent with the promoter sequence of GATA-1 listed as GenBank accession number AF196971 (FIG. 3). Subsequently, the plasmid obtained was cleaved at the NheI recognition site and the HindIII recognition site. The fragment obtained was inserted to the NheI/HindIII site of pGL3 basic, a vector for reporter gene assay comprising the firefly luciferase gene. Thus, the plasmid pGL3-IE was obtained, which has an 821-basepair sequence corresponding to the −789 to +32 region of the promoter region of the GATA-1 gene upstream of the firefly luciferase gene.

| | |
|---|---|
| ATAGATCTATCCCTGGCTCCCACCTCAG | (SEQ ID NO:32) |
| ATAAGCTTTGGTGGTTGCGGAGGGTTCG | (SEQ ID NO:33) |

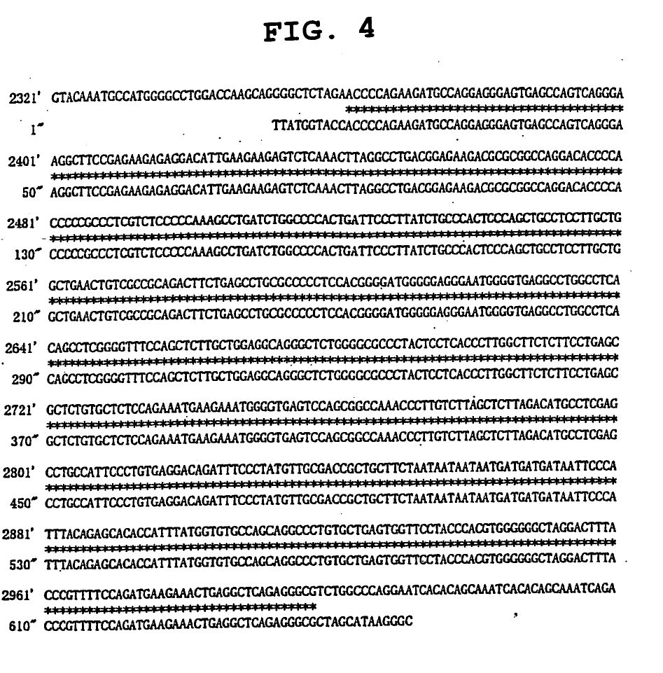

Subsequently, a 637-basepair fragment corresponding to the −3769 to −3133 region upstream of the transcription initiation site of human GATA-1 gene was obtained by the PCR method with human genomic DNA as a template. PCR was performed using the primers shown by SEQ ID NO:34 and SEQ ID NO:35 below. These primers were designed on the basis of the human GATA-1 gene sequence listed as gene database GenBank accession number AF196971. Additionally, a restriction endonuclease recognition site was added to the end of each primer to insert to a vector for reporter gene assay. The 637-basepair fragment obtained has a KpnI recognition site upstream of the promoter, and an NheI recognition site downstream of the promoter. The amplified 637-basepair fragment was inserted to the cloning vector pCR4. Subsequently, the base sequence of the inserted region in the plasmid obtained (SEQ ID NO:26) was confirmed. As a result, the base sequence of the inserted region (SEQ ID NO:26) was consistent with the promoter sequence of GATA-1 listed as GenBank accession number AF196971 (FIG. 4). Furthermore, the plasmid obtained was cleaved at the KpnI recognition site and the NheI recognition site. The fragment obtained was inserted to the KpnI/NheI site of the pGL3-IE promoter. Thus, the plasmid pGL3-HSI-IE Pro was obtained, which has a 637-basepair sequence corresponding to the −3769 to −3133 region upstream of the transcription initiation site of the GATA-1 gene, and an 821-basepair sequence corresponding to the −789 to +32 region, upstream of the firefly luciferase gene.

| | |
|---|---|
| ATGGTACCACCCCAGAAGATGCCAGGAG | (SEQ ID NO:34) |
| ATGCTAGCGCCCTCTGAGCCTCAGTTTC | (SEQ ID NO:35) |

### Example 3

### 3.1. Construction of Plasmids for Subcloning of HDAC1 to 8

Using the templates and primers shown in Table 1, various full-length HDAC isozymes were amplified by PCR and once subcloned into pGEM-T (Promega). Subsequently, each of full-length HDAC1 to 6 was inserted to pBluescriptII KS(+) (TOYOBO) via treatment with a restriction endonuclease (EcoRI/NotI for HDAC1, BamHI/NotI for HDAC2, EcoRI/NotI for HDAC3, EcoRI/NotI for HDAC4, HindIII/NotI for HDAC5, HindIII/NotI for HDAC6). Each of full-length HDAC7 and 8 was subcloned into pUC18 (TaKaRa) (SmaI treatment). For details, see Table 1.

TABLE 1

Preparation of subcloning plasmid of HDAC1-8

Template for

| | | | |
|---|---|---|---|
| amplifying a | GenBank | Size of | Name of |

| | | | | | |
|---|---|---|---|---|---|
| HDAC | full-length | Sequences of a pair of primers for amplifying | accession | PCR | plasmid |

| | | | | | |
|---|---|---|---|---|---|
| isozyme | HDAC | a full-length HDAC | number¹⁾ | product²⁾ | prepared³⁾ |

| | | | | | | |
|---|---|---|---|---|---|---|
| HDAC1 | Jurkat T- | HDAC1-E: | | D50405 | about 1.5 | pMH107 |
| | cell cDNA | GAGGAATTCAAGATGGCGCAGAC | (SEQ ID NO:36) | | kbp | |
| | library | HDAC1-N: | | | | |
| | (Clontech) | GGAGCGGCCGCTTCAGGCCAACTTG | (SEQ ID NO:37) | | | |
| HDAC2 | Human brain | HDAC2-B: | | HSU31814 | about 1.5 | pMH111 |
| | cDNA library | GGGGATCCATGGCGTACAGTCAAG | (SEQ ID NO:38) | | kbp | |
| | (Clontech) | HDAC2-N: | | | | |
| | | GGTGCGGCCGCCAAATTCAGGGGTTGCTG | (SEQ ID NO:39) | | | |
| HDAC3 | Human | HDAC3-E: | | U66914 | about 1.2 | pMH108 |
| | placenta | CCGGATCCACCATGGCCAAGAC | (SEQ ID NO:40) | | kbp | |
| | cDNA | HDAC3-N: | | | | |
| | library | GCAGCGGCCGCCACTCTTAAATCTCCAC | (SEQ ID NO:41) | | | |
| | (Clontech) | | | | | |
| HDAC4 | Human brain | HDAC4-E: | | AF132607 | About 3.2 | pMH106 |
| | cDNA library | GGGAATTCATGAGCTCCCAAAGCCATCC | (SEQ ID NO:42) | | kbp | |
| | (Clontech) | HDAC4-N: | | | | |
| | | AAGGCGCCGCAGCTTCGAGGGAGTGCTAC | (SEQ ID NO:43) | | | |
| HDAC5 | Human brain | HDAC5-H: | | AF132608 | about 3.3 | pMH109 |
| | cDNA library | GGCAAGCTTATGAACTCTCCCAACGAG | (SEQ ID NO:44) | | kbp | |
| | (Stratagene) | HDAC5-N: | | | | |
| | | GGGCGGCCGCGTCACAGGGCAGGCTCCTG | (SEQ ID NO:45) | | | |
| HDAC6 | Human brain | HDAC6-H: | | AF132609 | about 3.6 | pMH110 |
| | cDNA library | GCGAAGCTTATGACCTCAACCGGCCAG | (SEQ ID NO:46) | | kbp | |
| | (Stratagene) | HDAC6-N: | | | | |
| | | GCATGCGGCCGCTTAGTGTGGGTGGGGCATATC | (SEQ ID NO:47) | | | |
| HDAC7 | Human kidney | HDAC7/forward: | | AF239243 | about 2.6 | pUC18- |
| | cDNA library | CGAATTCAGCCGCAGCCCATGGACCT | (SEQ ID NO:48) | | kbp | HDAC7 |
| | (Clontech) | HDAC7/reverse: | | | | |
| | | CTTTACCCTGTGCACCCGGATCACGG | (SEQ ID NO:49) | | | |
| HDAC8 | Human lung | HDAC8/forward: | | HSA277724 | about 1.1 | pUC18- |
| | cDNA library | CGAATTCTTTAAGCGGAAGATGGAGG | (SEQ ID NO:50) | | kbp | HDAC8 |
| | (Clontech) | HDAC8/reverse: | | | | |
| | | AGGTACCGACCACATGCTTCAGATTC | (SEQ ID NO:51) | | | |

¹⁾GenBank accession number of various HDAC as reference in designing primer
²⁾Size of various full-length HDAC
³⁾Name of plasmids prepared for each HDAC. Subcloning plasmids for HDAC1-6 are pBluescriptII KS(+), and those for HDAC7 and 8 are pUC18.

3.2. Construction of Plasmids for Expression of HDAC1 to 8

Subsequently, plasmids for expression having a Flag sequence added to the C-terminal of each of HDAC1 to 8 were prepared. Details are shown below. HDAC3

Full-length HDAC3 was amplified by PCR with pMH108 as a template using the following primers 108E (SEQ ID NO:52) and 108KFN (SEQ ID NO:53). Meanwhile, the primers were designed to add a Flag sequence (DYKDDDDK) (SEQ ID NO:54) to the C-terminal. The PCR fragment obtained was subcloned into pGEM-T (Promega) to yield pGEM-HDAC3-Flag (E108KFN). E108KFN was inserted to pEAK10 (Edge BioSystems) via EcoRI/NotI (about 1.2 kbp fragment) treatment to yield pEAK-HDAC3 (pMH118).

| | | |
|---|---|---|
| 108E: | CTGAATTCACCATGGCCAAGACCG | (SEQ ID NO:52) |
| 108KFN: | GGGCGGCCGCCTACTTGTCATCGTCGT | (SEQ ID NO:53) |
| | CCTTGTAATCGGTACCAATCTCCACAT | |
| | CGCTTTC | |

HDAC1

Full-length HDAC1 was amplified by PCR with pMH107 as a template using the primers HDAC1-E (SEQ ID NO:36) and 107K (SEQ ID NO:55). Meanwhile, the primers were designed by modifying the C-terminal stop codon to insert a KpnI site. The PCR fragment obtained was subcloned into pGEM-T (Promega) to yield pGEM-HDAC1 (E1O7K). E107K was inserted to pMH118 (EcoRI/KpnI, about 6 kbp) via EcoRI/KpnI (about 1.5 kbp fragment) treatment to yield pEAK-HDAC1 (pMH119).

| | | |
|---|---|---|
| 107K: | ATCGGTACCGGCCAACTTGACCTCCTCCT | (SEQ ID NO: 55) |
| | TG | |

HDAC2

Full-length HDAC2 was amplified by PCR with pMH111 as a template using the primers HDAC2-B (SEQ ID NO:38) and 111K (SEQ ID NO:56). Meanwhile, the primers were designed by modifying the C-terminal stop codon to insert a KpnI site. The PCR fragment obtained was subcloned into pGEM-T (Promega) to yield pGEM-HDAC2. pGEM-HDAC2 was inserted to pFLAG-CMV-5a (SIGMA) (BamHI/KpnI) via BamHI/KpnI treatment to yield pFLAG-CMV-5a-HDAC2 (pMH113). pMH113 was inserted to pMH118 (EcoRI/KpnI, about 6 kbp) via EcoRI/KpnI (about 1.5 kbp fragment) treatment to yield pEAK-HDAC2 (pMH121).

| | | |
|---|---|---|
| 111K: | ATCGGTACCGGGGTTGCTGAGCTGTTCTG | (SEQ ID NO:56) |

Full-length HDAC4 was amplified by PCR with pMH106 as a template using the primers HDAC4-E (SEQ ID NO:42) and 106B (SEQ ID NO:57). Meanwhile, the primers were designed by modifying the C-terminal stop codon to insert a BglII site. The PCR fragment obtained was subcloned into pGEM-T (Promega) to yield pGEM-HDAC4 (E1O6B). A linker oligo having a BglII site and a FLAG sequence (DYKDDDDK) (FLAG-E (SEQ ID NO:58), prepared by 2o annealing FLAG-N (SEQ ID NO:59)) was inserted to an EcoRI/NotI-treated fragment (about 3 kbp) of pBluescriptII KS (+) to yield pBlue-Flag. E106B was inserted to pBlue-Flag via EcoRI/BglII treatment to yield pBKS-HDAC4 (E106BN). E106BN was inserted to about 6 kbp pEAK10 (Edge Byo Systems) (EcoRI/NotI) via EcoRI/NotI (about 3.2 kbp fragment) treatment to yield pEAK-HDAC4 (pMH122).

| | | |
|---|---|---|
| 106B: | GATAGATCTCAGGGGCGGCTCCTCTTC | (SEQ ID NO:57) |
| FLAG-E: | AATTCCTGCAGAGATCTGATTACAAGG | (SEQ ID NO:58) |
| | ACGACGATGACAAGTAGGC | |
| FLAG-N: | GGCGCCTACTTGTCATCGTCGTCCTTG | (SEQ ID NO:59) |
| | TAATCAGATCTCTGCAGG | |

HDAC5

A HindIII/NotI fragment (about 3.3 kbp) of pMH109 was inserted to p3XFLAG-CMV-10 (SIGMA) (HindIII/NotI, about 6.3 kbp) to yield p3XFLAG-CMV-10-HDAC5 (pMH144).

HDAC6

A HindIII/NotI fragment (about 3.6 kbp) of pMH110 was inserted to p3XFLAG-CMV-10 (SIGMA) (HindIII/NotI, about 6.3 kbp) to yield p3XFLAG-CMV-10-HDAC6 (pMH145).

HDAC7

A EcoRI/KpnI fragment (about 2.6 kbp) of pUC18-HDAC7 was inserted to PMH118 (EcoRI/KpnI, about 6 kbp) to yield pEAK-HDAC7 (pMH141).

HDAC8

A EcoRI/KpnI fragment (about 1.1 kbp) of pUC18-HDAC8 was inserted to PMH118 (EcoRI/KpnI, about 6 kbp) to yield pEAK-HDAC8 (pMH140).

For details about the individual HDAC expression vectors prepared, see Table 2.

TABLE 2
Preparation of HDAC1-8 expression plasmid

| | | Position of | |
|---|---|---|---|
| | | Flag tag | Name of |
| | | (DYKDDDK) | plasmid |
| HDAC | Expression plasmid used¹⁾ | for Each HDAC | prepared |
| HDAC1 | pEAK10 (Edge Bio Systems) | C-terminal | pMH119 |
| HDAC2 | pEAK10 (Edge Bio Systems) | C-terminal | pMH121 |
| HDAC3 | pEAK10 (Edge Bio Systems) | C-terminal | pMH118 |
| HDAC4 | pEAK10 (Edge Bio Systems) | C-terminal | pMH122 |
| HDAC5 | p3XFLAG-CMV-10 (SIGMA) | N-terminal | pMH144 |
| HDAC6 | p3XFLAG-CMV-10 (SIGMA) | N-terminal | pMH145 |
| HDAC7 | pEAK10 (Edge Bio Systems) | C-terminal | pMH141 |
| HDAC8 | pEAK10 (Edge Bio Systems) | C-terminal | pMH140 |

¹⁾pEAK10 (Edge Bio Systems) is regulated by EF1α (elongation factor 1α) promoter. p3XFLAG-CMV-10 (SIGMA) is regulated by CMV (cytomegarovirus) promoter.
In addition, HDAC1, 2, 3, 4, 7 and 8 are inserted to each expression plasmid via EcoRI/NotI, and HDAC5 and 6 are inserted to each expression plasmid via HindIII/NotI.

### Example 4

### Effects of Overexpression of Various HDAC Isozymes on IL-2 Transcriptional Activity

1 μg of the plasmid pGL3 IL2 Pro43, which has a human IL-2 promoter sequence (the −674 to +54 region in the vicinity of the transcription initiation site) upstream of the firefly luciferase gene, 6 μg of pRL-TK (Promega), and 10 μg of each of various HDAC isozyme expression plasmids or a mock plasmid (the mock plasmid was pEAK10, and the various HDAC expression plasmids were pMH119 for HDAC1, pMH121 for HDAC2, pMH118 for HDAC3, pMH122 for HDAC4, pMH144 for HDAC5, pMH145 for HDAC6, pMH141 for HDAC7, and pMH140 for HDAC8, respectively) were mixed, and 1×10⁷ Jurkat cells (ATCC, TIB-152) were transformed by the electroporation method (BIO-RAD, Gene Pulser II, voltage 300 V, charge 975 pF, 400 μL).

After transformation, 2.5 mL of RPMI 1640 (SIGMA) containing 10% FBS (MOREGATE) (10% FBS RPMI 1640) was added, and this mixture was dispensed to a 96-well white plate in a ratio of 50 μL/well. After cultivation under conditions of 37° C., 5% CO₂ and saturated humidity for 10 hours, a 10% FBS RPMI 1640 solution was added at 50 μL/well, and a liquid mixture of Phorbol 12-Myristate 13-Acetate (PMA, SIGMA), Ionomycin (SIGMA) and Anti-CD28 antibody (Pharmingen) in 10% FBS RPMI 1640 was added at 50 μL/well (the final concentrations were 50 ng/mL, 1 μg/mL and 75 ng/mL, respectively) to stimulate the Jurkat cells. After cultivation under conditions of 37° C., 5% CO₂ and saturated humidity for 12 hours, luciferase activity in the cells was measured according to the manual of the Dual Luciferase Assay System (Promega) using a multi-label counter (1420 MULTILABEL COUNTER ARVO SX, WALLAC).

As a result, it was found that luciferase was induced from pGL3 IL2 Pro43 in response to the stimulation of the Jarkat cells by PMA, Ionomycin, and Anti-CD28 antibody, and that the expression level thereof increased with the overexpression of HDAC4 and 8 (FIG. 5). It was also confirmed using the Cell Counting Kit-8 (DOJIN) that the overexpression of various HDAC isozymes did not significantly affect the degree of cell proliferation (FIG. 5).

These results demonstrate that HDAC4 and 8 are HDAC isozymes involved in the IL-2 transcription system.

### Example 5

### Effects of Overexpression of Various HDAC Isozymes on GATA-1 Transcriptional Activity

5 μg of the plasmid pGL3-HSI-IE Pro, which has a human GATA-1 promoter sequence (the −3769 to −3133 and −789 to +32 regions in the vicinity of the transcription initiation site) upstream of the firefly luciferase gene, and 10 μg of each of various HDAC isozyme expression plasmids or a mock plasmid (the mock plasmid was pEAK10, and HDAC1 to 4, 7 and 8 were under the control of the EF-1α promoter, HDAC5 and 6 were under the control of the CMV promoter, and the various HDAC isozymes had a Flag tag at the C-terminal or N-terminal thereof) were mixed, and 8.75×10⁶ HEL cells (accession number JCRB0062, JCRB) were transformed by the electroporation method (BIO-RAD, Gene Pulser II, voltage 1750 V, charge 10 μF, 365 gL). After transformation, 2 mL of 10% FBS RPMI 1640 was added, and this mixture was dispensed to a 96-well white plate in a ratio of 50 μL/well. After cultivation under conditions of 37° C., 5% CO₂ and saturated humidity for 3 hours, a 10% FBS RPMI 1640 medium was added at 100 μL/well. After cultivation under conditions of 37° C., 5% CO₂ and saturated humidity for 8 hours, luciferase activity in the cells was measured according to the manual of the Dual Luciferase Assay System using a multi-label counter.

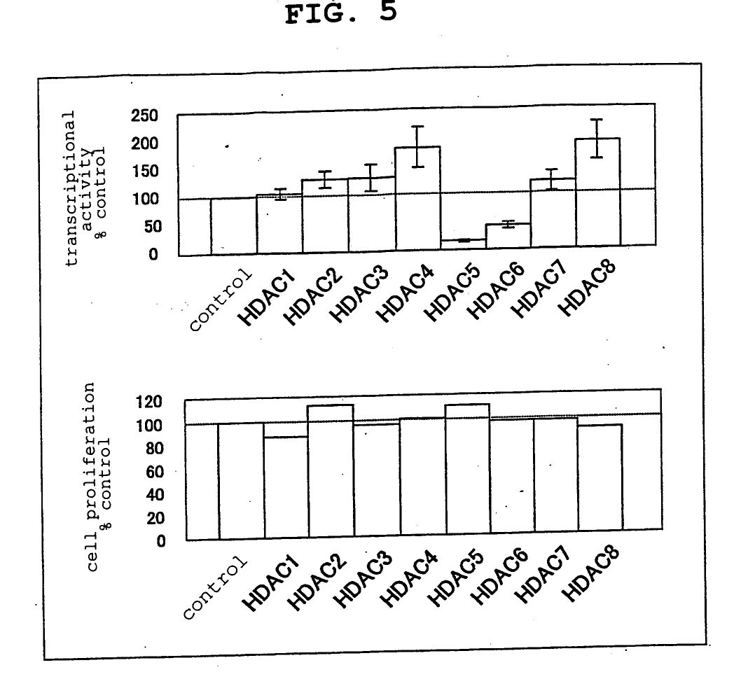

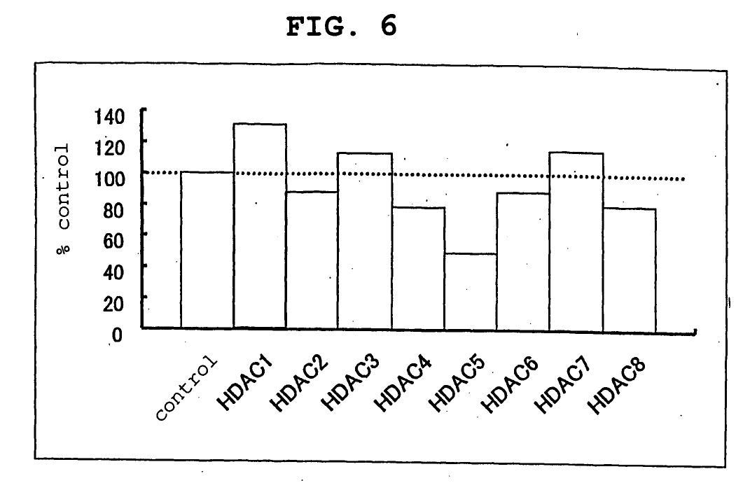

As a result, it was found that luciferase depending on the GATA-1 promoter was induced from pGL3-HSI-IE Pro, and that the expression level of the induced luciferase increased slightly with the overexpression of HDAC1, 3 and 7 (FIG. 6).

These results demonstrate that HDAC1, 3 and 7 are involved in the GATA-1 transcription system, and that HDAC4 and 8, which are involved in the IL-2 transcription system, are not significantly involved in the GATA-1 transcription system.

### Example 6

### Preparation of HDAC4 Dominant Negative Variants

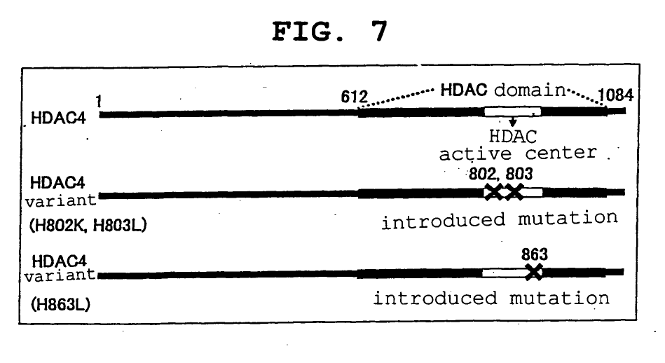

For HDAC4, which seems to be largely involved in IL-2 transcription, HDAC4 dominant negative variants were prepared for the purpose of losing the function of endogenous HDAC4. Hence, from the literature information in PNAS, 1998, Vol. 95, p.3519-3524 & Mol. Cell. Biol., 1999, Vol. 19 (11), p.7816-7827, the sites for mutagenesis to reduce HDAC activity were determined (H802K, H803L, H863L) (FIG. 7). The human HDAC4 CDS base sequence was obtained from GenBank accession number AF132607, and primers for mutagenesis were designed (for the base sequences of the individual primers, see Table 3; for the HDAC4 CDS base sequence, see SEQ ID NO:1; for the amino acid sequence encoded by the HDAC4 CDS base sequence, see SEQ ID NO:2).

TABLE 3

Preparation of HDAC4 dominant negative variant (templates and
primers for PCR reaction)

HDAC4
dominant

| | |
|---|---|
| negative | size of PCR |

| | | | |
|---|---|---|---|
| variant | template | sequence of a pair of primers | product |

| | | | | |
|---|---|---|---|---|
| (HB02K, | (1) E106BN | (1) NdeIFW: | | (1) about 140 bp |
| HB03L) | | CATGACACCATATGGAACGAGG | (SEQ ID NO:60) | |
| | | HDAC4-mutation-2RV: | | |
| | | GCAAGCTTTCCAGGGGGGCG | (SEQ ID NO:61) | |
| | (2) E106BN | (2) HDAC4-mutation-2Fw: | | (2) about 180 bp |
| | | CCCCCTGGAAGCTTGCGGA | (SEQ ID NO:62) | |
| | | BsrGIRV: | | |
| | | GACATGTACAGGACGCTAGGGT | (SEQ ID NO:63) | |
| | (3) PCR | (3) NdeIFW: | | (3) about 300 bp |
| | fragment | CAGTGACACCATATGGAACGAGG | (SEQ ID NO:60) | |
| | of (1) and | BsrGIRV: | | |
| | (2) | GACATGTACAGGACGCTAGGGT | (SEQ ID NO:63) | |
| (H863L) | E106BN | mutation3FW: | | about 700 bp |
| | | CCTGTACATGTCCCTCCTTCG | (SEQ ID NO:64) | |
| | | 3′XbaBg1Rv: | | |
| | | ATAGATCTAGACAGGGGCGG | (SEQ ID NO:65) | |

A PCR reaction was conducted according to the protocol of Expand (High fidelity PCR System (Roche)). E106BN, which was used as a template, is a plasmid prepared by inserting full-length HDAC4 CDS to pBluescriptII KS (+) (TOYOBO) (EcoRI/NotI, the initiation codon was located on the EcoRI side).

Using the Topo TA cloning kit for sequence (Invitrogen Company), each of the PCR products obtained ((3) for H802K and H803L variants) was subcloned into the TOPO cloning vector for sequence (pCR4-HDAC4 (H802K, H803L) and pCR4-HDAC4 (H863L)). Subsequently, a PCR fragment confirmed as being mutated by sequencing was inserted to an HDAC4 expression plasmid (pMH122) by the procedures shown below.

HDAC4 Dominant Negative Variants (H802K, H803L)

An NdeI/BsrGI-treated fragment (about 300 bp) of pCR4-HDAC4 (H802K, H803L) was inserted to E106BN (NdeI/BsrGI) to yield pBKS-HDAC4 (H802K, H803L). Subsequently, an EcoRI/NotI-treated fragment (about 3.3 kbp) of pBKS-HDAC4 (H802K, H803L) was inserted to pMH122 (EcoRI/NotI, about 6 kbp) to yield pEAK-HDAC4 (H802K, H803L). The base sequence in the coding region of HDAC4 dominant negative variants (H802K, H803L) is shown by SEQ ID NO:3, and the amino acid sequence thereof is shown by SEQ ID NO:4.

HDAC4 dominant negative variant (H863L)

A BsrGI/BglII-treated fragment (about 750 bp) of pCR4-HDAC4 (H863L) was inserted to E106BN (BsrGI/BglII, about 3 kbp) to yield pBKS-HDAC4 (H863L). Subsequently, an EcoRI/NotI-treated fragment (about 3.3 kbp) of pBKS-HDAC4 (H863L) was inserted to pMH122 (EcoRI/NotI, about 6 kbp) to yield pEAK-HDAC4 (H863L). The base sequence in the coding region of HDAC4 dominant negative variant (H863L) is shown by SEQ ID NO:5, and the amino acid sequence thereof is shown by SEQ ID NO:6.

### Example 7

### Preparation of HDAC1 Dominant Negative Variants

HDAC1 dominant negative variants were prepared in the same manner as the preparation of the HDAC4 dominant negative variants. The human HDAC1 CDS base sequence was obtained from GenBank accession number D50405, and primers for mutagenesis were designed (for the base sequences of the individual primers, see Table 4; for the base sequence of HDAC1 CDS, see SEQ ID NO:9; for the amino acid sequence encoded by the HDAC1 CDS base sequence, see SEQ ID NO:10).

TABLE 4

Preparation of HDAC1 dominant negative variants (templates and primers
for PCR reaction)

HDAC1
dominant

| | |
|---|---|
| negative | size of PCR |

| | | | |
|---|---|---|---|
| variant | template | sequence of a pair of primers | product |

| | | | | |
|---|---|---|---|---|
| (H140K, | (1) pEAK-HDAC1 | (1) HDAC1-E: | | |
| H141L) | | GAGGAATTCAAGATGGCGCAGAC | (SEQ ID NO:66) | (1) about 440 bp |
| | | 1-140KL-RV: | | |
| | | CTCGGACTTCTTTGCAAGCTTCAGG | (SEQ ID NO:67) | |
| | (2) pEAK-HDAC1 | (2) 1-140KL-FW: | | |
| | | GGGCCTGAAGCTTGCAAAGAAGTC | (SEQ ID NO:68) | (2) about 120 bp |
| | | 1-BsrGI-RV: | | |
| | | GTGAATATCAATGTCAATGTACAGC | (SEQ ID NO:69) | |
| | (3) PCR fragment | (3) HDAC1-E: | | |
| | of (1) and (2) | GAGGAATTCAAGATGGCGCAGAC | (SEQ ID NO:66) | (3) about 530 bp |
| | | 1-BsrGI-RV: | | |
| | | GTGAATATCAATGTCAATGTACAGC | (SEQ ID NO:69) | |
| (H199L) | (1) pEAK-HDAC1 | (1) 1-StuI-FW: | | |
| | | GACGGCGTGGAAGAGGCCTTC | (SEQ ID NO:70) | (1) about 80 bp |
| | | 1-199L-RV: | | |
| | | GAAGTACTCTCCATACTTAAGAAAGG | (SEQ ID NO:71) | |
| | (2) pEAK-HDAC1 | (2) 1-199L-FW: | | |
| | | CTGTGTCCTTTCTTAAGTATGGAGAG | (SEQ ID NO:72) | (2) about 100 bp |
| | | 1-HpaI-RV: | | |
| | | TCTCGGAGCGGGTAGTTAACAG | (SEQ ID NO:73) | |
| | (3) PCR fragment | (3) 1-StuI-FW: | | |
| | of (1) and (2) | GACGGCGTGGAAGAGGCCTTC | (SEQ ID NO:70) | (3) about 150 bp |
| | | 1-HpaI-RV: | | |
| | | TCTCGGAGCGGGTAGTTAACAG | (SEQ ID NO:73) | |

A PCR reaction was conducted according to the protocol of Expand (High fidelity PCR System (Roche)). pEAK-HDAC1 (pMH119), which was used as a template, is a plasmid prepared by inserting full-length HDAC1 CDS to pEAK10.

Using the Topo TA cloning kit for sequence (Invitrogen Company), each of the PCR products obtained (3) was subcloned into the TOPO cloning vector for sequence (pCR4-HDAC1 (H14OK, H141L) and pCR4-HDAC1 (H199L)). A PCR fragment confirmed as being mutated by sequencing was inserted to an HDAC1 expression plasmid (pMH119) using the procedures shown below. E107K is a plasmid prepared by inserting full-length HDAC1 CDS to pGEM-T (Promega) (EcoRI/KpnI, the initiation codon is located on the EcoRI side).

HDAC1 Dominant Negative variants (H140K, H141L)

An EcoRI/BsrGI-treated fragment (about 530 bp) of pCR4-HDAC1 (H140K, H141L) was inserted to E107K (EcoRI/BsrGI, about 3 kbp) to yield pGEM-HDAC1 (H140K, H141L). Subsequently, pGEM-HDAC1 (H140K, H141L) (EcoRI/KpnI, about 1.5 kbp) was inserted to an EcoRI/KpnI-treated fragment (about 5.9 kbp) of pMH119 to yield pEAK-HDAC1 (H140K, H141L). The base sequence in the coding region of HDAC1 dominant negative variants (H140K, H141L) is shown by SEQ ID NO:11, and the amino acid sequence thereof is shown by SEQ ID NO:12.

HDAC1 Dominant Negative Variant (H199L)

An StuI/HpaI-treated fragment (about 150 bp) of pCR4-HDAC1 (H199L) was inserted to E107K (StuI/HpaI) (about 3 kbp) to yield pGEM-HDAC1 (H199L). Subsequently, pGEM-HDAC1 (H199L) (EcoRI/KpnI, about 1.5 kbp) was inserted to an EcoRI/KpnI-treated fragment (about 5.9 kbp) of pMH119 to yield pEAK-HDAC1 (H199L). The base sequence in the coding region of HDAC1 dominant negative variant (H199L) is shown by SEQ ID NO:13, and the amino acid sequence thereof is shown by SEQ ID NO:14.

### Example 8

### Preparation of HDAC3 Dominant Negative Variants

HDAC3 dominant negative variants were prepared in the same manner as the preparation of the HDAC4 dominant negative variants. The human HDAC3 CDS base sequence was obtained from GenBank accession number U66914, and primers for mutagenesis were designed (for the base sequences of the individual primers, see Table 5; for the HDAC3 CDS base sequence, see SEQ ID NO:15; for the amino acid sequence encoded by the HDAC3 CDS base sequence, see SEQ ID NO:16).

TABLE 5

Preparation of HDAC3 dominant negative variants (templates and primers
for PCR reaction)

HDAC3
dominant

| | |
|---|---|
| negative | size of PCR |

| | | | |
|---|---|---|---|
| variant | template | sequence of a pair of primers | product |

| | | | | |
|---|---|---|---|---|
| (H134K, | (1) pEAK-HDAC3 | (1) 3-BglII-FW: | | |
| H135L) | | AACCCAGCTGAACAACAAGATCTG | (SEQ ID NO:74) | (1) about 75 bp |
| | | 3-134KL-RV: | | |
| | | CTCAAACTTCTTGGCAAGCTTCAGAC | (SEQ ID NO:75) | (2) about 140 bp |
| | (2) pEAK-HDAC3 | (2) 3-134KL-FW: | | |
| | | TGGTGGTCTGAAGCTTGCCAAGAAG | (SEQ ID NO:76) | (3) about 180 bp |
| | | 3-NcoI-RV: | | |
| | | CCCGTCACCATGGTGGATGTC | (SEQ ID NO:77) | |
| | (3) PCR | (3) 3-BglII-FW: | | |
| | fragment of | AACCCAGCTGAACAACAAGATCTG | (SEQ ID NO:74) | |
| | (1) and (2) | 3-NcoI-RV: | | |
| | | CCCGCACCATGGTGGATGTC | (SEQ ID NO:77) | |
| (H193L) | (1) pEAK-HDAC3 | (1) 3-NcoI-FW: | | |
| | | TGACATTGACATCCACCATGTG | (SEQ ID NO:78) | (1) about 100 bp |
| | | 3-193L-RV: | | |
| | | GAAGTAATTTCCGTACTTAAGGAAGG | (SEQ ID NO:79) | (2) about 190 bp |
| | (2) pEAK-HDAC3 | (2) 3-193L-FW: | | |
| | | TGTCCTTCCTTAAGTACGGAAATTAC | (SEQ ID NO:80) | (3) about 260 bp |
| | | 3-PmaCI-RV: | | |
| | | GAGCACAATGCACGTGGGTTGG | (SEQ ID NO:81) | |
| | (3) PCR | (3) 3-NcoI-FW: | | |
| | fragment of | TGACATTGACATCCACCATGGTG | (SEQ ID NO:78) | |
| | (1) and (2) | 3-PmaCI-RV: | | |
| | | GAGCACAATGCACGTGGGTTGG | (SEQ ID NO:81) | |

A PCR reaction was conducted according to the protocol of Expand (High fidelity PCR System (Roche)). pEAK-HDAC3 (pMH118), which was used as a template, is a plasmid prepared by inserting full-length HDAC3 CDS to pEAK10.

Using the Topo TA cloning kit for sequence (Invitrogen Company), each of the PCR products (3) obtained was subcloned into the TOPO cloning vector for sequence (pCR4-HDAC3 (H134K, H135L) and pCR4-HDAC3 (H193L)). A PCR fragment confirmed as being mutated by sequencing was inserted to an HDAC3 expression plasmid (pMH118) by the procedures shown below.

HDAC3 Dominant Negative Variants (H134K, H135L)

An EcoRI/KpnI-treated fragment (about 1.3 kbp) of pMH118 was inserted to pBluescriptII KS (−) (TOYOBO) (EcoRI/KpnI) (about 3 kbp) to yield pBKS-HDAC3. Subsequently, pCR4-HDAC3 (H134K, H135L) (BglII/NcoI, about 180 bp) was inserted to pBKS-HDAC3 (BglII/NcoI, about 3 kbp) to yield pBKS-HDAC3 (H134K, H135L). Finally, pBKS-HDAC3 (H134K, H135L) (EcoRI/KpnI, about 1.3 kbp) was inserted to an EcoRI/KpnI-treated fragment (about 5.7 kbp) of pMH118 to yield pEAK-HDAC3 (H134K, H135L). The base sequence in the coding region of HDAC3 dominant negative variants (H134K, H135L) is shown by SEQ ID NO:17, and the amino acid sequence thereof is shown by SEQ ID NO:18.

HDAC3 Dominant Negative Variant (H193L)

A pCR4-HDAC3 (H134K, H135L) (NcoI/PmaCI, About 260 bp) was inserted to pMH118 (NcoI/PmaCI, about 7 kbp) to yield pEAK-HDAC3 (H193L). The base sequence in the coding region of HDAC3 dominant negative variant (H193L) is shown by SEQ ID NO:19, and the amino acid sequence thereof is shown by SEQ ID NO:20.

### Example 9

### IL-2 Transcriptional Activity Suppression by Expression of HDAC4 Dominant Negative Variants

To 1 μg of the plasmid pGL3 IL2 Pro43, which has a human IL-2 promoter sequence (the −674 to +54 region in the vicinity of the transcription initiation site) upstream of the firefly luciferase gene, and 6 μg of pRL-TK (Promega), the plasmids for the expression of wild-type HDAC4 or each of HDAC4 dominant negative variants (H802K, H803L, H863L) prepared in Example 3 was mixed in various ratios (1, 3 and 10 μg), and 1×10⁷ Jurkat cells (ATCC, TIB-152) were transformed by the electroporation method (BIO-RAD, Gene Pulser II, voltage 300 V, charge 975 μF, 400 μL).

After transformation, 2.5 mL of RPMI 1640 containing 10% FBS (MOREGATE) (10% FBS RPMI 1640) was added, and this mixture was dispensed to a 96-well white plate in a ratio of 50 μL/well. After cultivation under conditions of 37° C., 5% CO₂ and saturated humidity for 10 hours, a liquid mixture of Phorbol 12-Myristate 13-Acetate (PMA, SIGMA), Ionomycin (SIGMA) and Anti-CD28 antibody (Pharmingen) in 10% FBS RPMI 1640 was added at 50 μL/well (the final concentrations were 50 ng/mL, 1 μg/mL and 75 ng/mL, respectively) to stimulate the Jurkat cells. After cultivation under conditions of 37° C., 5% CO₂ and saturated humidity for 12 hours, luciferase activity in the cells was measured according to the manual of the Bright-Glo Luciferase Assay System (Promega) using a multi-label counter (1420 MULTILABEL COUNTER ARVO SX, WALLAC).

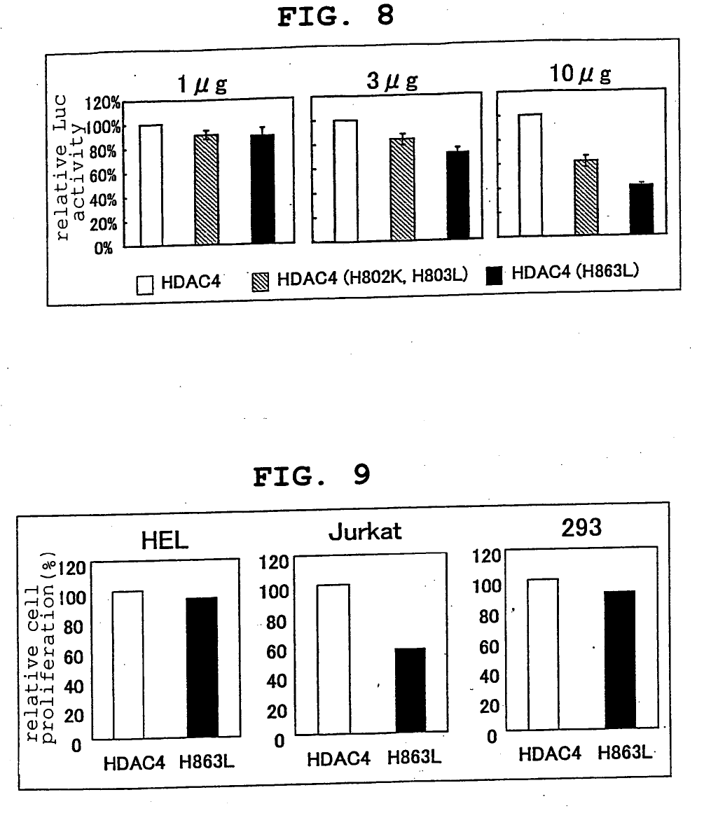

As a result, IL-2 transcription suppression depending on the expression level of HDAC4 dominant negative variants (H802K, H803L, H863L) was observed (FIG. 8). The expression level of the transferred HDAC was confirmed by Western blotting using an anti-Flag antibody (SIGMA, anti-Flag M2).

These results suggest that IL-2 transcription may be suppressed by selectively suppressing the function of HDAC4.

### Example 10

### Cell Proliferation Suppression by Expression of HDAC4 Dominant Negative Variants

10 μg of a plasmid for the expression of wild-type HDAC4 and the HDAC4 dominant negative variant (H863L) prepared in Example 6 was transfected to Jurkat cells, HEL cells and 293 cells.

1×10⁷ Jurkat cells (ATCC, TIB-152) were transformed by the electroporation method (BIO-RAD, Gene Pulser II, voltage 300 V, charge 975 μF, 400 μL). After transformation, 2.5 mL of RPMI 1640 containing 10% FBS (MOREGATE) (10% FBS RPMI 1640) was added, and this mixture was dispensed to a 96-well white plate in a ratio of 50 μL/well. After cultivation under conditions of 37° C., 5% CO₂ and saturated humidity for 5 hours, a liquid mixture of Phorbol 12-Myristate 13-Acetate (PMA, SIGMA), Ionomycin (SIGMA) and Anti-CD28 antibody (Pharmingen) in 10% FBS RPMI 1640 was added at 50 μL/well (the final concentrations were 50 ng/mL, 1 μg/mL and 75 ng/mL, respectively) to stimulate the Jurkat cells. After cultivation under conditions of 37° C., 5% CO₂ and saturated humidity for 4 hours, the number of cell was confirmed using the Cell Counting Kit-8 (DOJIN) 8.75×106 HEL cells (accession number JCRBOO62, JCRB) were transformed by the electroporation method (BIO-RAD, Gene Pulser II, voltage 1750 V, charge 10 μF, 365 μL). After transformation, 2 mL of 10% FBS RPMI 1640 was added, and this mixture was dispensed to a 96-well white plate in a ratio of 50 μL/well. After cultivation under conditions of 37° C., 5% CO₂ and saturated humidity for 10 hours, 10% FBS RPMI 1640 medium was added at 50 μL/well, further cultivation was conducted for 12 hours, and the number of cell was confirmed using the Cell Counting Kit-8 (DOJIN).

293 cells (ATCC, CRL-1573) were transfected by the calcium phosphate method (a mixture of DNA and calcium phosphate was added to 293 cells in culture using a DMEM (SIGMA) medium containing 10% FBS, and the medium was replaced with a fresh one 6 hours later); after cultivation under conditions of 37° C., 5% CO₂ and saturated humidity for 48 hours, the number of cell was confirmed using the Cell Counting Kit-8 (DOJIN).

As a result, cell proliferation inhibition specific for Jurkat cells due to the expression of HDAC4 dominant negative variant (H863L) was observed (FIG. 9). For each type of cells, the expression level of the transferred HDAC was confirmed by Western blotting using an anti-Flag antibody (SIGMA, anti-Flag M2).

These results demonstrate that the proliferation of T cell lineage is selectively suppressed by selectively inhibiting the function of HDAC4.

### Example 11

### Effects of expression of HDAC1, 3 and 4 Dominant Negative Variants on IL-2 and GATA-1 Transcription Activities

IL-2 and GATA-1 transcription activities were measured with the expression of the HDAC1, 3 and 4 dominant negative variants prepared in Examples 6, 7 and 8.

1×10⁷ Jurkat cells (ATCC, TIB-152) were transformed with 10 μg of a plasmid for the expression of various HDACs or HDAC variants by the electroporation method (BIO-RAD, Gene Pulser II, voltage 300 V, charge 975 μF, 400 μL). After transformation, 2.5 mL of RPMI 1640 (SIGMA) containing 10% FBS (MOREGATE) (10% FBS RPMI 1640) was added, and this mixture was dispensed to a 96-well white plate in a ratio of 50 μL/well. After cultivation under conditions of 37° C., 5% CO₂ and saturated humidity for 10 hours, a liquid mixture of Phorbol 12-Myristate 13-Acetate (PMA, SIGMA), Ionomycin (SIGMA) and Anti-CD28 antibody (Pharmingen) in 10% FBS RPMI 1640 was added at 50 μL/well (the final concentrations were 50 ng/mL, 1 μg/mL and 75 ng/mL, respectively) to stimulate the Jurkat cells. After cultivation under conditions of 37° C., 5% CO₂ and saturated humidity for 12 hours, luciferase activity in cells were measured according to the manual of the Bright-Glo Luciferase Assay System (Promega), and IL-2 transcriptional activity was measured using a multi-label counter (1420 MULTILABEL COUNTER ARVO SX, WALLAC).

Using 15 μg of pGL3-HSI-IE Pro and 10 μg of a plasmid for the expression of various HDACs or HDAC variants, 8.75×10⁶ HEL cells (accession number JCRB0062, JCRB) were transformed by the electroporation method (BIO-RAD, Gene Pulser II, voltage 1750 V, charge 10 μF, 365 μL). After transformation, 2 mL of 10% FBS RPMI 1640 was added, and this mixture was dispensed to a 96-well white plate in a ratio of 50 μL/well. After cultivation under conditions of 37° C., 5% CO₂ and saturated humidity for 3 hours, 10% FBS RPMI 1640 medium was added at 50 μL/well. After cultivation under conditions of 37° C., 5% CO₂ and saturated humidity for 8 hours, luciferase activity in cells were measured according to the manual of the Bright-Glo Luciferase Assay System, and GATA-1 transcriptional activity was measured using a multi-label counter.

As a result, it was found that IL-2 transcription suppression was observed only with the expression of HDAC4 dominant negative variants (H802K, H803L, H863L), and that GATA-1 transcription suppression was not observed at that time (FIG. 10). For each type of cells, the expression level of the transferred HDAC was confirmed by Western blotting using an anti-Flag antibody (SIGMA, anti-Flag M2).

These results suggest that immune function can be suppressed, without suffering thrombocytopenia, by selectively inhibiting the function of HDAC4.

### Example 12

### Complex Formation of HDAC4

In Mol. Cell, Vol. 9, p.45-57, 2002, it is reported that Class II HDACs have formed a complex with N-CoR and HDAC3, and that HDAC activity with histone as a substrate is much more portent for HDAC3 than for Class II HDACs. Hence, the formation of complexes of crudely purified Class II HDACs was confirmed.

7 μg of each of HDAC4, 5 and 7 expression plasmids was transfected to PEAK Rapid cells (Edge BioSystems) by the calcium phosphate method; 48 hours later, whole cell lysate (cells were disrupted in 25 mM Tris, pH 7.4, 150 mM NaCl, 1 mM CaCl₂, 1% Triton X-100, and cell debris was removed by centrifugation) was crudely purified using an anti-Flag antibody column (SIGMA, anti-Flag M2-agarose) (washed with TBS, then eluted with 0.1M Glysine-HCl, pH 3.5). Subsequently, the crudely purified fraction was subjected to SDS-PAGE and then stained with CBB.

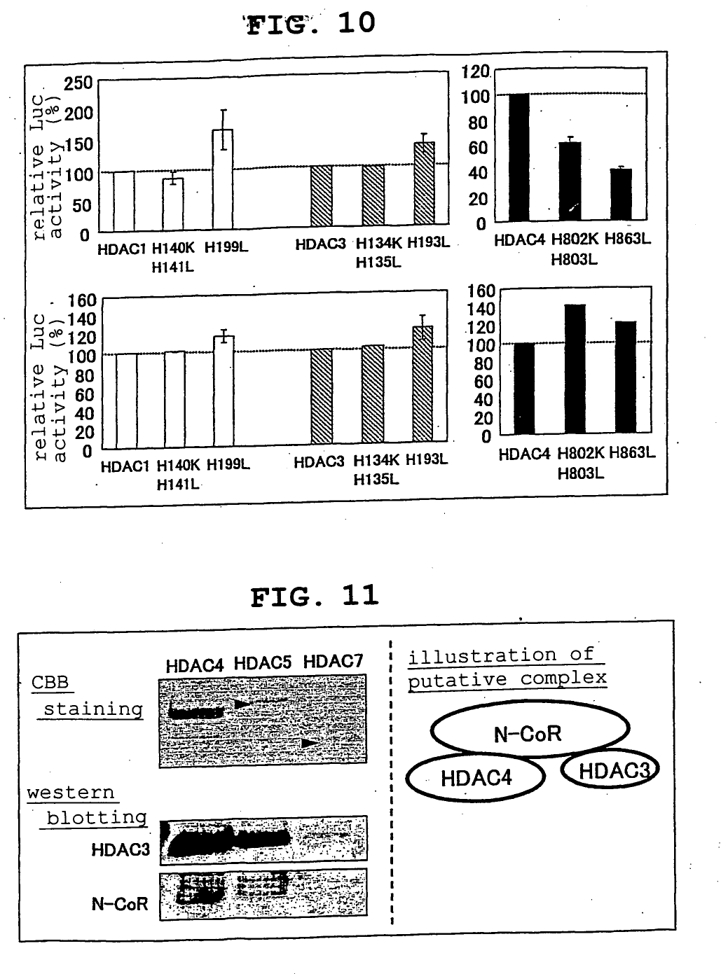

As a result, the expression of the transferred HDAC4, 5 and 7 was confirmed (FIG. 11). It was also confirmed by Western blotting that each of the crudely purified fractions of HDAC4, 5 and 7 contained HDAC3 and N-COR (using an anti-HDAC3 antibody of SIGMA Company and an anti-N-CoR antibody of Santa Cruz Company).

These results confirm that the crudely purified HDAC4, 5 and 7 have formed a complex with N-CoR and HDAC3.

### Example 13

### Complex Forming Ability of HDAC4 Dominant Negative Variants

In Mol. Cell, Vol. 9, p.45-57, 2002, it is reported that some HDAC4 variants do not form a complex with N-CoR and HDAC3. Hence, the complex forming ability of HDAC4 dominant negative variant (H863L) was examined.

7 μg of a plasmid for the expression of wild-type HDAC4 and HDAC4 dominant negative variant (H863L) was transfected to PEAK Rapid cells (Edge BioSystems) by the calcium phosphate method; 48 hours later, whole cell lysate (cells were disrupted in 25 mM Tris, pH 7.4, 150 mM NaCl, 1 mM CaCl₂, 1% Triton X-100, and cell debris was removed by centrifugation) was crudely purified using an anti-Flag antibody column (SIGMA, anti-Flag M2-agarose) (washed with TBS, then eluted with 0.1M Glysine-HCl, pH 3.5). Subsequently, the crudely purified fraction was subjected to SDS-PAGE and then stained with CBB.

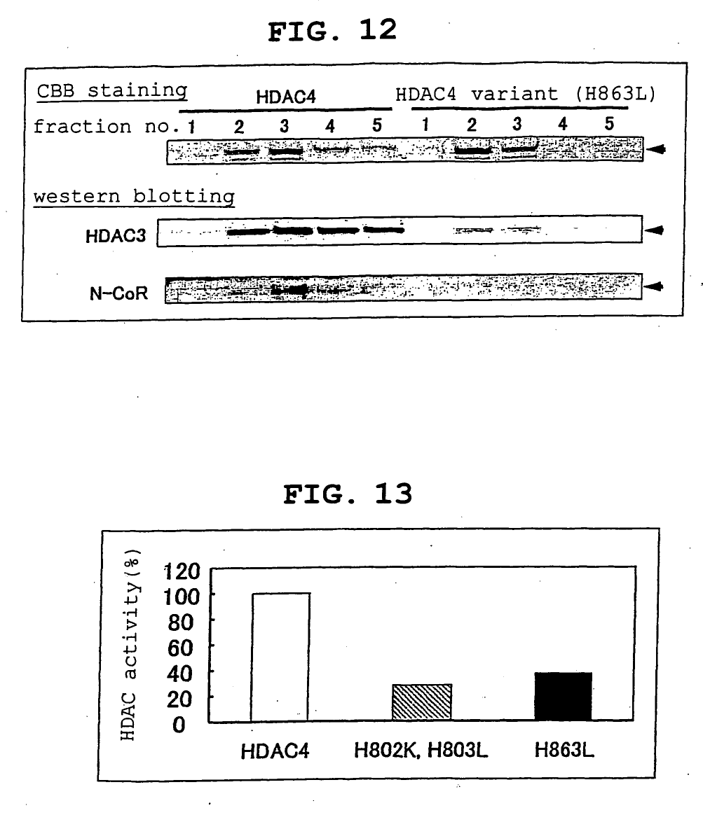

As a result, the expression of the transferred HDAC4 and HDAC4 dominant negative variant (H863L) was confirmed (FIG. 12). It was also confirmed by Western blotting (using an anti-HDAC3 antibody of SIGMA Company and an anti-N-CoR antibody of Santa Cruz Company) that the crudely purified fraction of HDAC4 contained HDAC3 and N-CoR, and that the crudely purified fraction of HDAC4 dominant negative variant (H863L) contained almost no HDAC3 or N-COR.

These results demonstrate that HDAC4 dominant negative variant (H863L) has a decreased complex forming ability with N-COR and HDAC3 compared to wild-type HDAC4.

### Example 14

### Measurement of HDAC Activity

In Mol. Cell, Vol. 9, p.45-57, 2002, it is reported that HDAC4 variants which decreased complex forming ability with N-CoR and HDAC3 shows decrease of HDAC activity with histone as a substrate. Hence, the HDAC activities of HDAC4 dominant negative variants (H802K, H803L, H863L) with histone as a substrate were measured.

7 μg of a plasmid for the expression of wild-type HDAC4 and HDAC4 dominant negative variants (H802K, H803L, H863L) was transfected to 293T cells by the calcium phosphate method; 48 hours later, whole cell lysate (cells were disrupted in 25 mM Tris, pH 7.4, 150 mM NaCl, 1 mM CaCl₂, 1% Triton X-100, and cell debris was removed by centrifugation) was crudely purified using an anti-Flag antibody column (SIGMA, anti-Flag M2-agarose) (washed with TBS, then eluted with 0.1M Glysine-HCl, pH 3.5), and used for the measurement of HDAC activity. The reaction substrate used was a [³H]-labeled acetylated histone prepared by cultivating Jurkat cells (2×10⁸ cells) in 40 mL of an RPMI (SIGMA) medium containing 300 MBq [³H] sodium acetate (NENTM Life Science Products, Inc.) for 2 hours, disrupting the cells in Lysis buffer, pH 6.5 (10 mM Tris-HCl, 50 mM Sodium Hydrogen sulfate, 1% Triton X-100, 10 mM MgCl₂-6H₂O, 8.6% Sucrose), washing the cells with Wash buffer (10 mM Tris-HCl, 13 mM EDTA), and then adding 0.4N H₂SO₄.

As a result, the HDAC4 dominant negative variants (H802K, H803L, H863L) had decreased HDAC activity compared to wild-type HDAC4 (FIG. 13).

These results confirm that the HDAC4 dominant negative variants (H802K, H803L, H863L) have decreased complex forming ability with N-CoR and HDAC3 (Example 13), and hence decreased HDAC activity with histone as a substrate.

### Example 15

### Construction of Detection System For Interaction of HDAC4 and N-CoR

Since it was found that an HDAC4 dominant negative variant (H863L) had decreased complex forming ability with N-CoR compared to wild-type HDAC4, it was considered that the same IL-2 transcription suppressive action as that obtained with the expression of HDAC4 dominant negative variant is exhibited by inhibiting the interaction of HDAC4 and N-CoR.

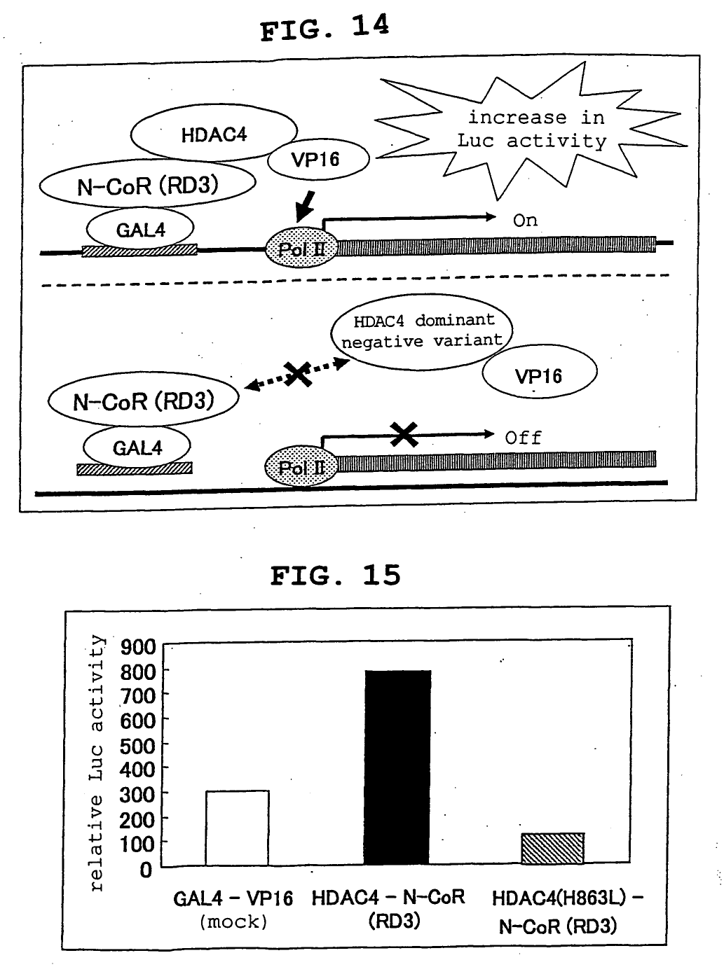

Hence, to construct a screening system for a drug that inhibits the interaction of HDAC4 and N-CoR, the CheckMate Mammalian Two-Hybrid System (Promega) was used (FIG. 14).

First, from the literature information in Mol. Cell, 2002, Vol. 9, p.45-57, the site corresponding to the RD3 sequence of human N-COR was determined (1005-1498a.a.). The human N-COR CDS base sequence was obtained from GenBank accession number AF044209, and human N-CoR(RD3) cDNA was amplified from human-lung-derived cDNA by the PCR method. The primers used were RD3-Bam-FW (SEQ ID NO:82) and RD3-Not-RV (SEQ ID NO:83) below. For the base sequence of human N-CoR(RD3), see SEQ ID NO:21; for the amino acid sequence encoded by the base sequence of human N-CoR (RD3), see SEQ ID NO:22. The PCR fragment obtained was confirmed by sequencing (for N-CoR(RD3), a variation of L1014V was observed, but this was adopted since it was reported as a variant in SWISS-PROT), and it was inserted to pBIND (Promega) via treatment with restriction endonuclease (BamHI/NotI) to yield a GAL4-N-CoR (RD3) expression plasmid. Also, HDAC4 or an HDAC4 dominant negative variant (H863L) in full length was inserted to pACT (Promega) via restriction endonuclease treatment (XbaI/NotI) to yield HDAC4-VP16 and HDAC4 (H863L)-VP16 expression plasmids.

| | | |
|---|---|---|
| RD3-Bam-FW: | GGGATCCGTGAAGTCCTTCAGCCT | (SEQ ID NO: 82) |
| | GCTCC | |
| RD3-Not-RV: | TGCGGCCGCCATGGGTGAGCCTCT | (SEQ ID NO: 83) |
| | GGACA | |

Subsequently, 10 μg of pG5-Luc (Promega), 3 μg of BND and 3 μg of pACT, or 10 μg of pG5-Luc, 3 μg of pBND-N-CoR (RD3) and 3 μg of pACT-HDAC4, or 10 μg of pG5-Luc, 3 μg of pBND-N-CoR(RD3) and 3 μg of pACT-HDAC4 (H863L), were mixed respectively, and each mixture was transfected to 1×10⁷ Jurkat cells (ATCC, TIB-152) by the electroporation method (BIO-RAD, Gene Pulser II, voltage 300 V, charge 975 μF, 400 μL). Twenty-two hours after transfection, luciferase activity in the cells was measured according to the manual of the Bright-Glo Luciferase Assay System (Promega) using a multi-label counter (1420 MULTILABEL COUNTER ARVO SX, WALLAC).

As a result, GAL4 transcription activation due to the interaction of N-CoR (RD3) and HDAC4 was detected, and it was confirmed that the HDAC4 dominant negative variant (H863L) did not interact with N-CoR(RD3) (FIG. 15).

From these results, it was evaluated that a detection system for the interaction of HDAC4 and N-CoR (RD3) was constructed.

### Example 16

### Effects of HDAC4-Selective Gene Expression Suppression Using siRNA on IL-2 and GATA-1 Transcription Activities

A mixture of four kinds of HDAC4-targeted siRNA, i.e., siGENE HDAC4 SMARTpool (manufactured by Dharmacon Inc., marketed by B-Bridge international Inc., code number M-003497-00-05), or Non-specific Control Duplex IX (47% GC content) (manufactured by Dharmacon Inc., marketed by B-Bride international Inc., code number D-001206-09-20), and pGL3 IL2 Pro43 were transfected to Jurkat cells by the electroporation method using the same conditions as those mentioned above, and IL-2 transcriptional activity was measured. Likewise, siGENE HDAC4 SMARTpool or Non-specific Control Duplex IX, and pGL3-HSI-IE Pro were transfected to HEL cells, and GATA-1 transcriptional activity was measured. The conditions of siRNA transfection and their effects in Jurkat cells and HEL cells were previously confirmed using pGL3-control (Promega) and Luciferase GL3 Duplex (B-Bride: D-001400-01-20).

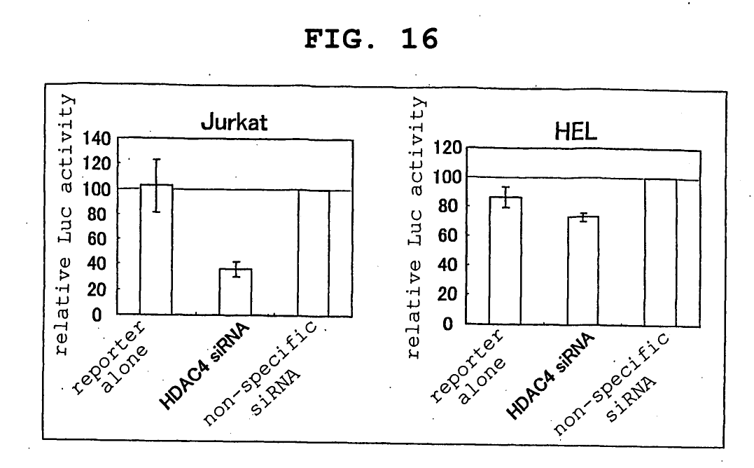

As a result, IL-2 transcription suppression due to the selective suppression of the expression of the HDAC4 gene was observed, but GATA-1 transcription suppression was not observed (FIG. 16).

These results again suggest that immune function can be suppressed, without suffering thrombocytopenia, by selectively inhibiting the function of HDAC4.

### INDUSTRIAL APPLICABILITY

The present invention has shown that immunity can be suppressed, IL-2 production can be inhibited, or immunocyte proliferation can be suppressed, without exhibiting a noticeable thrombocytopenic activity and/or GATA-1 production inhibitory activity, by selectively suppressing HDAC4/ HDAC8, out of various HDACs. Therefore, by selecting a compound that selectively suppresses HDAC4/ HDAC8, it is possible to select an immunosuppressant having low thrombocytopenic activity or an IL-2 production inhibitor or an immunocyte proliferation inhibitor having low GATA-1 production inhibitory activity more conveniently and in a shorter time than conventional. Hence, the method of the present invention is very useful for research for developing new drug.

The present application is base on Japanese patent application no. 2002-378800 filed in Japan on Dec. 27, 2002 and its content is herein incorporated by reference.

What is claimed is:

1. An agent for inhibiting IL-2 production and/or an agent for inhibiting immunocyte proliferation, each of which having low GATA-1 production inhibitory activity, comprising a selective HDAC4 and/or HDAC8 inhibitor.

2. An agent for suppressing immunity having low thrombocytopenic activity, comprising a selective HDAC4 and/or HDAC8 inhibitor.

3. A method of selecting an IL-2 production inhibitor and/or an immunocyte proliferation inhibitor which have low GATA-1 production inhibitory activity, which comprises measuring the HDAC4 and/or HDAC8 enzyme inhibitory activity of a test substance.

4. A method of selecting an immunosuppressant having low thrombocytopenic activity, which comprises measuring the HDAC4 and/or HDAC8 enzyme inhibitory activity of a test substance.

5. A method of selecting an immunosuppressant having low thrombocytopenic activity, which comprises steps (i) and (ii) below:
(i) measuring the HDAC4 and/or HDAC8 enzyme inhibitory activity of a test substance, and
(ii) measuring the HDAC enzyme inhibitory activity of the test substance, wherein the HDAC enzyme inhibitory activity is one or more HDAC enzyme inhibitory activities selected from a group consisting of HDAC1, HDAC2, HDAC3, HDAC5, HDAC6 and HDAC7 enzyme inhibitory activities.

6. The method of claim 3, which is a method of selecting an IL-2 production inhibitor and/or an immunocyte proliferation inhibitor which have low GATA-1 production inhibitory activity, and which comprises steps (i) to (iii) below, wherein the IL-2 production inhibitor and/or the immunocyte proliferation inhibitor selectively inhibits HDAC4 and/or HDAC8 enzyme activity:
(i) expressing each of the HDAC1, HDAC2, HDAC3, HDAC4, HDAC5, HDAC6, HDAC7 and HDAC8 genes to obtain respective enzyme solutions;
(ii) measuring HDAC enzyme activity for each of the enzyme solutions in the presence of a test substance;
(iii) selecting a test substance that selectively suppresses HDAC enzyme activity only when using an enzyme solution obtained by expressing the HDAC4 gene or the HDAC8 gene.

7. The method of claim 4, which is a method of selecting an immunosuppressant having low thrombocytopenic activity, and which comprises steps (i) to (iii) below, wherein the immunosuppressant selectively inhibits HDAC4 and/or HDAC8 enzyme activity:
(i) expressing each of the HDAC1, HDAC2, HDAC3, HDAC4, HDAC5, HDAC6, HDAC7 and HDAC8 genes to obtain respective enzyme solutions;
(ii) measuring.HDAC enzyme activity for each of the enzyme solutions in the presence of a test substance;
(iii) selecting a test substance that selectively suppresses HDAC enzyme activity only when using an enzyme solution obtained by expressing the HDAC4 gene or the HDAC8 gene.

8. The method of claim 4, which is a method of selecting an immunosuppressant having low thrombocytopenic activity, and which comprises steps (i) to (iii) below, wherein the immunosuppressant selectively inhibits HDAC4 and/or HDAC8 enzyme activity:
(i) measuring HDAC enzyme activity for an enzyme solution partially purified from human cells, in the presence of a test substance;
(ii) measuring HDAC enzyme activity for an enzyme solution obtained by expressing the HDAC4 gene and/or the HDAC8 gene, in the presence of a test substance;
(iii) selecting a test substance that selectively suppresses HDAC enzyme activity only when using an enzyme solution obtained by expressing the HDAC4 gene and/or the HDAC8 gene, by comparing the enzyme activities measured in (i) and (ii).

9. A method of selecting an immunosuppressant having low thrombocytopenic activity, which comprises steps (i) to (iii) below, wherein an HDAC inhibitor that specifically inhibits the formation of a complex of HDAC4 and N-COR is selected from among test HDAC inhibitors:
(i) expressing the HDAC4 gene and the N-CoR gene to obtain an enzyme solution;
(ii) allowing the enzyme solution to coexist with a test HDAC inhibitor;
(iii) determining whether or not a complex of HDAC4 and N-CoR is formed in the mixture of enzyme solution and test HDAC inhibitor obtained in (ii).

10. A method of selecting an immunosuppressant having low thrombocytopenic activity, which comprises steps (i) and (ii) below, wherein an HDAC inhibitor that specifically inhibits the formation of a complex of the HDAC4 fusion protein comprising at least a portion of the HDAC4 protein, and the N-CoR fusion protein comprising at least a portion of the N-COR protein, is selected from among test HDAC inhibitors:
(i) expressing the gene that encodes the HDAC4 fusion protein and the gene that encodes the N-CoR fusion protein in cells;
(ii) determining whether or not the test HDAC inhibitor inhibits the formation of a complex of the HDAC4 fusion protein and the N-COR fusion protein.

11. A method of selecting a compound having low thrombocytopenic activity and having immunosuppressive activity, which comprises steps (i) and (ii) below, wherein an HDAC inhibitor that suppresses the expression level of HDAC4 and/or HDAC8 is selected from among test HDAC inhibitors:
(i) allowing cells that express HDAC4 and/or HDAC8 to coexist with a test HDAC inhibitors;
(ii) measuring the HDAC4 and/or HDAC8 expression level in the cells.

12. A method of selecting a compound having low thrombocytopenic activity and having immunosuppressive activity, which comprises steps (i) and (ii) below, wherein an HDAC inhibitor that selectively inhibits the binding of HDAC4 and/or HDAC8 and an HDAC4- and/or HDAC8-specific ligand is selected from among test HDAC inhibitors:
(i) expressing the HDAC4 and/or HDAC8 gene to obtain an enzyme solution;
(ii) measuring the binding activity of the HDAC4 and/or HDAC8 enzyme and the HDAC4- and/or HDAC8-specific ligand for the enzyme solution in the presence of an HDAC4- and/or HDAC8-specific ligand and a test HDAC inhibitor.

13. An assay kit for the selection of an immunosuppressant having low thrombocytopenic activity including. a selective HDAC4 and/or HDAC8 inhibitor, which includes at least (i) and (ii) below:
(i) enzyme solutions prepared by expressing each of the HDAC1, HDAC2, HDAC3, HDAC5, HDAC6 and HDAC7 genes;
(ii) an enzyme solution prepared by expressing each of the HDAC4 and/or HDAC8 gene.

14. A DNA having the base sequence that encodes the amino acid sequence shown by SEQ ID NO:4 or SEQ ID NO:6.

15. An HDAC4 variant that is the amino acid sequence shown by SEQ ID NO:4 or SEQ ID NO:6.

16. A method of inhibiting IL-2 production and/or immunocyte proliferation with low GATA-1 production inhibitory activity, which comprises administering an effective amount of selective HDAC4 and/or HDAC8 inhibitor to a subject.

17. A method of suppressing immunity with low thrombocytopenic activity, which comprises administering an effective amount of selective HDAC4 and/or HDAC8 inhibitor to a subject.

18. Use of a selective HDAC4 and/or HDAC8 inhibitor for the production of an agent for inhibiting IL-2 production and/or an agent for inhibiting immunocyte proliferation which have low GATA-1 production inhibitory activity.

19. Use of a selective HDAC4 and/or HDAC8 inhibitor for the production of an agent for suppressing immunity having low thrombocytopenic activity.


SEQUENCE LISTING (86 sequences)

SEQ ID NO: 1 - DNA, 3255 bp, Homo sapiens
  CDS (1)..(3255): human histone deacetylase-4 (HDAC4) gene
atgagctcccaaagccatccagatggactttctggccgagaccagccagtggagctgctgaatcctgcccgcgtgaaccacatgcccagcacggtggatgtggccacggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgctgagttccagaggcagcacgagcagctctcccggcagcacgaggcgcagctccacgagcacatcaagcaacaacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacaccgagagactcacccttcccgccctccagcagaggctctcccttttccccggcacccacctcactccctacctgagcacctcgcccttggagcgggacggaggggcagcgcacagccctcttctgcagcacatggtcttactggagcagccaccggcacaagcacccctcgtcacaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccctagcgtcctgtacatgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctgtag

SEQ ID NO: 2 - protein, 1084 aa, Homo sapiens
MSSQSHPDGLSGRDQPVELLNPARVNHMPSTVDVATALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDTERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 3 - DNA, 3255 bp, Artificial Sequence
  CDS (1)..(3255): Dominant negative mutant (H802K, H803L) of human histone deacetylase-4 (HDAC4) gene
atgagctcccaaagccatccagatggactttctggccgagaccagccagtggagctgctgaatcctgcccgcgtgaaccacatgcccagcacggtggatgtggccacggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgctgagttccagaggcagcacgagcagctctcccggcagcacgaggcgcagctccacgagcacatcaagcaacaacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacaccgagagactcacccttcccgccctccagcagaggctctcccttttccccggcacccacctcactccctacctgagcacctcgcccttggagcgggacggaggggcagcgcacagccctcttctgcagcacatggtcttactggagcagccaccggcacaagcacccctcgtcacaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggaaagcttgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccctagcgtcctgtacatgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcgtctctgtctctgatcgaggctcagacttgcgagaacgaagaagccgagacggtcaccgccatggcctcgctgtccgtgggcgtgaagcccgccgaaaagagaccagatgaggagcccatggaagaggagccgcccctgtag

SEQ ID NO: 4 - protein, 1084 aa, Artificial Sequence
  Dominant negative mutant (H802K, H803L) of human histone deacetylase-4 (HDAC4) gene
MSSQSHPDGLSGRDQPVELLNPARVNHMPSTVDVATALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDTERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGKLAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 5 - DNA, 3255 bp, Artificial Sequence
  CDS (1)..(3255): Dominant negative mutant (H863L) of human histone deacetylase-4 (HDAC4) gene
atgagctcccaaagccatccagatggactttctggccgagaccagccagtggagctgctgaatcctgcccgcgtgaaccacatgcccagcacggtggatgtggccacggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgctgagttccagaggcagcacgagcagctctcccggcagcacgaggcgcagctccacgagcacatcaagcaacaacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacaccgagagactcacccttcccgccctccagcagaggctctcccttttccccggcacccacctcactccctacctgagcacctcgcccttggagcgggacggaggggcagcgcacagccctcttctgcagcacatggtcttactggagcagccaccggcacaagcacccctcgtcacaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccctagcgtcctgtacatgtccctccttcgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctgtag

SEQ ID NO: 6 - protein, 1084 aa, Homo sapiens
  Dominant negative mutant (H863L) of human histone deacetylase-4 (HDAC4) gene
MSSQSHPDGLSGRDQPVELLNPARVNHMPSTVDVATALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDTERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLLRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 7 - DNA, 1743 bp, Homo sapiens
  CDS (43)..(1176): human histone deacetylase-8 (HDAC8) gene
gccagatctggaaggtggctgcggaacggttttaagcggaagatggaggagccggaggaaccggcggacagtgggcagtcgctggtcccggtttatatctatagtcccgagtatgtcagtatgtgtgactccctggccaagatccccaaacgggccagtatggtgcattctttgattgaagcatatgcactgcataagcagatgaggatagttaagcctaaagtggcctccatggaggagatggccaccttccacactgatgcttatctgcagcatctccagaaggtcagccaagagggcgatgatgatcatccggactccatagaatatgggctaggttatgactgcccagccactgaagggatatttgactatgcagcagctataggaggggctacgatcacagctgcccaatgcctgattgacggaatgtgcaaagtagcaattaactggtctggagggtggcatcatgcaaagaaagatgaagcatctggtttttgttatctcaatgatgctgtcctgggaatattacgattgcgacggaaatttgagcgtattctctacgtggatttggatctgcaccatggagatggtgtagaagacgcattcagtttcacctccaaagtcatgaccgtgtccctgcacaaattctccccaggatttttcccaggaacaggtgacgtgtctgatgttggcctagggaagggacggtactacagtgtaaatgtgcccattcaggatggcatacaagatgaaaaatattaccagatctgtgaaagtgtactaaaggaagtataccaagcctttaatcccaaagcagtggtcttacagctgggagctgacacaatagctggggatcccatgtgctcctttaacatgactccagtgggaattggcaagtgtcttaagtacatccttcaatggcagttggcaacactcattttgggaggaggaggctataaccttgccaacacggctcgatgctggacatacttgaccggggtcatcctagggaaaacactatcctctgagatcccagatcatgagtttttcacagcatatggtcctgattatgtgctggaaatcacgccaagctgccggccagaccgcaatgagccccaccgaatccaacaaatcctcaactacatcaaagggaatctgaagcatgtggtctagttgacagaaagagatcaggtttccagagctgaggagtggtgcctataatgaagacagcgtgtttatgcaagcagtttgtggaatttgtgactgcagggaaaatttgaaagaaattacttcctgaaaatttccaaggggcatcaagtggcagctggcttcctggggtgaagaggcaggcaccccagagtcctcaactggacctaggggaagaaggagatatcccacatttaaagttcttatttaaaaaaacacacacacacaaatgaaatttttaatctttgaaaattatttttaagcgaattggggaggggagtattttaatcatcttaaatgaaacagatcagaagctggatgagagcagtcaccagtttgtagggcaggaggcagctgagaggcagggtttgggcctcaggaccatccaggtggagccctgggagagagggtactgatcagcagactgggaggtggggagaagtccgctggtgttgttttagtgttatatatctttggtttttttaataaaatctttgaaaacctaaaaaaaaaaaaaaaaaaaaaaaaaaaaaa

SEQ ID NO: 8 - protein, 377 aa, Homo sapiens
MEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVV

SEQ ID NO: 9 - DNA, 1449 bp, Homo sapiens
  CDS (1)..(1449): human histone deacetylase-1 (HDAC1) gene
atggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgcaggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctga

SEQ ID NO: 10 - protein, 482 aa, Homo sapiens [sequence omitted]

SEQ ID NO: 11 - DNA, 1449 bp, Artificial Sequence
  CDS (1)..(1449): Dominant negative mutant (H140K, H141L) of human histone deacetylase-1 (HDAC1) gene
atggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgaagcttgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgcaggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctga

SEQ ID NO: 12 - protein, 482 aa, Artificial Sequence
  Dominant negative mutant (H140K, H141L) of human histone deacetylase-1 (HDAC1) gene
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLKLAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCRTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 13 - DNA, 1449 bp, Artificial Sequence
  CDS (1)..(1449): Dominant negative mutant (H199L) of human histone deacetylase-1 (HDAC1) gene
atggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcttaagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgcaggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctga

SEQ ID NO: 14 - protein, 482 aa, Artificial Sequence
  Dominant negative mutant (H199L) of human histone deacetylase-1 (HDAC1) gene
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFLKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCRTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 15 - DNA, 1287 bp, Homo sapiens
  CDS (1)..(1287): human histone deacetylase-3 (HDAC3) gene
atggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccaacatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatcctccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatttaa

SEQ ID NO: 16 - protein, 428 aa, Homo sapiens [sequence omitted]

SEQ ID NO: 17 - DNA, 1287 bp, Artificial Sequence
  CDS (1)..(1287): Dominant negative mutant (H134K, H135L) of human histone deacetylase-3 (HDAC3) gene
atggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccaacatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgaagcttgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatcctccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatttaa

SEQ ID NO: 18 - protein, 428 aa, Artificial Sequence
  Dominant negative mutant (H134K, H135L) of human histone deacetylase-3 (HDAC3) gene
MAKTVAYFYDPDVGNFHYGAGHPMKPHRLALTHSLVLHYGLYKKMIVFKPYQASQHDMCRFHSEDYIDFLQRVSPTNMQGFTKSLNAFNVGDDCPVFPGLFEFCSRYTGASLQGATQLNNKICDIAINWAGGLKLAKKFEASGFCYVNDIVIGILELLKYHPRVLYIDIDIHHGDGVQEAFYLTDRVMTVSFHKYGNYFFPGTGDMYEVGAESGRYYCLNVPLRDGIDDQSYKHLFQPVINQVVDFYQPTCIVLQCGADSLGCDRLGCFNLSIRGHGECVEYVKSFNIPLLVLGGGGYTVRNVARCWTYETSLLVEEAISEELPYSEYFEYFAPDFTLHPDVSTRIENQNSRQYLDQILQTIFENLKMLNHAPSVQIHDVPADLLTYDRTDEADAEERGPEENYSRPEAPNEFYDGDHDNDKESDVEI

SEQ ID NO: 19 - DNA, 1287 bp, Artificial Sequence
  CDS (1)..(1287): Dominant negative mutant (H193L) of human histone deacetylase-3 (HDAC3) gene
atggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccaacatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccttaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatcctccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatttaa

SEQ ID NO: 20 - protein, 428 aa, Artificial Sequence
  Dominant negative mutant (H193L) of human histone deacetylase-3 (HDAC3) gene
MAKTVAYFYDPDVGNFHYGAGHPMKPHRLALTHSLVLHYGLYKKMIVFKPYQASQHDMCRFHSEDYIDFLQRVSPTNMQGFTKSLNAFNVGDDCPVFPGLFEFCSRYTGASLQGATQLNNKICDIAINWAGGLHHAKKFEASGFCYVNDIVIGILELLKYHPRVLYIDIDIHHGDGVQEAFYLTDRVMTVSFLKYGNYFFPGTGDMYEVGAESGRYYCLNVPLRDGIDDQSYKHLFQPVINQVVDFYQPTCIVLQCGADSLGCDRLGCFNLSIRGHGECVEYVKSFNIPLLVLGGGGYTVRNVARCWTYETSLLVEEAISEELPYSEYFEYFAPDFTLHPDVSTRIENQNSRQYLDQILQTIFENLKMLNHAPSVQIHDVPADLLTYDRTDEADAEERGPEENYSRPEAPNEFYDGDHDNDKESDVEI

SEQ ID NO: 21 - DNA, 1458 bp, Homo sapiens
  CDS (1)..(1458): human N-CoR (RD3) gene
gaagtccttcagcctgctccacatcaagtgataactaatctccctgaaggcgttcggcttccgacaactcgaccaaccaggccaccgccccctctcatcccgtcatccaaaaccacagtggcttcagaaaaaccatcttttataatgggaggctccatctcacagggaacaccaggcacttatttgacttctcataatcaggcttcctacactcaagaaacacccaagccgtcagtaggatctatctctcttggactgccacggcaacaggaatctgccaaatcagctactttgccctacatcaagcaggaagaattttctccccgaagccaaaactcacaacctgagggtctgttggtcagggcccaacatgaaggtgtagtcagaggtaccgcaggagccatacaagaaggaagtataactcggggaactccaaccagcaaaatttcagtggagagcattccatccctacggggctctatcactcagggcaccccggctctgccccagactggcataccaacagaggctttggtgaaggggtccatttcgagaatgcccattgaagacagcagtcctgagaaaggcagagaggaagctgcatccaaaggccatgttatttatgaaggcaaaagtggacatatcttgtcatatgataatattaagaatgcccgagaagggactaggagtccaagaacagctcatgaaatcagtttaaagagaagctatgaatcagtggaaggaaatataaagcaagggatgtcaatgagggagtctcctgtatcagcaccgttagaggggctgatatgccgagcattacccagggggagtcctcattctgacctcaaagaaaggactgtattgtctggctccataatgcaggggacaccaagagcaacaactgaaagctttgaagatggccttaaatatcccaaacaaattaaaagggaaagtcctcccatacgagcatttgaaggtgccattaccaaaggaaaaccatatgatggcatcaccaccatcaaagaaatggggcgttccattcatgagattccaaggcaagatattttaactcaggaaagtcggaaaactccagaagtggtccagagcacacggccgataattgagggttccatttcccagggcacaccaataaagtttgacaacaactcaggtcaatctgccatcaaacacaatgtcaaatccttaatcacggggcctagcaaactatcccgtggaatgcctccgctggaaattgtgccagagaacataaaagtggtagaacggggaaaatatgaggatgtgaaagcaggcgagaccgtgcgttcccggcacacgtcagtggtaagctctggcccctccgttcttaggtccacactgcatgaagctcccaaagcacaactgagccctgggatttatgatgacaccagtgcacggaggacccctgtgagttatcaaaac

SEQ ID NO: 22 - protein, 486 aa, Homo sapiens
EVLQPAPHQVITNLPEGVRLPTTRPTRPPPPLIPSSKTTVASEKPSFIMGGSISQGTPGTYLTSHNQASYTQETPKPSVGSISLGLPRQQESAKSATLPYIKQEEFSPRSQNSQPEGLLVRAQHEGVVRGTAGAIQEGSITRGTPTSKISVESIPSLRGSITQGTPALPQTGIPTEALVKGSISRMPIEDSSPEKGREEAASKGHVIYEGKSGHILSYDNIKNAREGTRSPRTAHEISLKRSYESVEGNIKQGMSMRESPVSAPLEGLICRALPRGSPHSDLKERTVLSGSIMQGTPRATTESFEDGLKYPKQIKRESPPIRAFEGAITKGKPYDGITTIKEMGRSIHEIPRQDILTQESRKTPEVVQSTRPIIEGSISQGTPIKFDNNSGQSAIKHNVKSLITGPSKLSRGMPPLEIVPENIKVVERGKYEDVKAGETVRSRHTSVVSSGPSVLRSTLHEAPKAQLSPGIYDDTSARRTPVSYQN

SEQ ID NO: 23 - DNA, 731 bp, Homo sapiens
  misc_feature (1)..(731): 5′-flanking region of Human interleukin-2 (IL-2) gene
ctgagtatttaacaatcgcaccctttaaaaaatgtacaatagacattaagagacttaaacagatatataatcattttaaattaaaatagcgttaaacagtacctcaagctcaataagcattttaagtattctaatcttagtatttctctagctgacatgtaagaagcaatctatcttattgtatgcaattagctcattgtgtggataaaaaggtaaaaccattctgaaacaggaaaccaatacacttcctgtttaatcaacaaatctaaacatttattcttttcatctgtttactcttgctcttgtccaccacaatatgctattcacatgttcagtgtagttttaggacaaagaaaattttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccacaa

SEQ ID NO: 24 - DNA, 527 bp, Homo sapiens
  misc_feature (73)..(506): 5′-flanking region of Human interleukin-2 (IL-2) gene
  misc_feature (4)..(9): a or g or c or t/u, unknown or other
  misc_feature (12): a or g or c or t/u, unknown or other
  misc_feature (56): a or g or c or t/u, unknown or other
gggnnnnnnggngatcctctaccactatagggcgaattgaatttagcggccgcganttcgcccttcgctagctgctcttgtccaccacaatatgctattcacatgttcagtgtagttttaggacaaagaaaattttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccacaagcttcgaagggcgaattcgtt

SEQ ID NO: 25 - DNA, 900 bp, Homo sapiens
  misc_feature (57)..(875): Promoter region of Human GATA-1 gene
tgcctctttactatagggcgaattgaatttagcggccgcgaattcgcccttgatctatccctggctcccacctcagtttcccgcctccaaggcagcatggcgggcaagaagttgaggccactgtccctgggtgttcctacccccacaccctcaccccaagacagcctgttactgcggcgccaacagccacggtcgcctacatctgataagacttatctgctgccccagggcaggccggagctggcgtaagccccagtggggcgctaagtgagtgtgcccctgcctcccgccagcactggcctggcctgcaggcttagcctgggtcatcaaggtatcccacaggctctagttcaaatccagcagaacctctctgagcctcactcttctcacctgcaaaatgggtacagccacatcccttctctccctgcagccaggaagacgcacatacacaggagtctagcccacaccggccccgcacaaattaagggctttactctctgaaaagcccagtgaagtcatgaaaccatatctgctattttcatttatcttggtttcagcctattttgcttgtctggacactacagtccacgggagcctaggtcgagcgaggtccaagaatccccagggtgggcagggagggtggaagagggcctccagtgcccaagaggtgccccacaagcatgggacccgccccctcccctggactgccccacccactggggcaccagccactccctggggaggagggaggagggagaagggagggagggagggagggaggaagggagcctcaaaggccaaggccagccaggacaccccctgggatcacactgagcttgccacatccccaaggcggccgaaccctccgcaaccaccaaagcttataagggcgaattcgtt

SEQ ID NO: 26 - DNA, 660 bp, Homo sapiens
  misc_feature (11)..(647): Promoter region of Human GATA-1 gene
ttatggtaccaccccagaagatgccaggagggagtgagccagtcagggaaggcttccgagaagagaggacattgaagaagagtctcaaacttaggcctgacggagaagacgcgcggccaggacaccccacccccgccctcgtctcccccaaagcctgatctggccccactgattcccttatctgcccactcccagctgcctccttgctggctgaactgtcgccgcagacttctgagcctgcgccccctccacggggatgggggagggaatggggtgaggcctggcctcacagcctcggggtttccagctcttgctggaggcagggctctggggcgccctactcctcacccttggcttctcttcctgagcgctctgtgctctccagaaatgaagaaatggggtgagtccagcggccaaacccttgtcttagctcttagacatgcctcgagcctgccattccctgtgaggacagatttccctatgttgcgaccgctgcttctaataataataatgatgatgataattcccatttacagagcacaccatttatggtgtgccagcaggccctgtgctgagtggttcctacccacgtggggggctaggactttacccgttttccagatgaagaaactgaggctcagagggcgctagcataagggc

SEQ ID NO: 27 - DNA, 32 bp, Artificial Sequence
  PCR primer for amplifying the 5′-flanking region of Human interleukin-2 (IL-2) gene
tcgctagcctgagtatttaacaatcgcaccct

SEQ ID NO: 28 - DNA, 30 bp, Artificial Sequence
  PCR primer for amplifying the 5′-flanking region of Human interleukin-2 (IL-2) gene
cgaagcttgtggcaggagttgaggttactg

SEQ ID NO: 29 - DNA, 777 bp, Homo sapiens
  misc_feature (1)..(777): Corresponding to the sequence (+661 to +1437) in the GenBank database (Acession HSIL05)
atcagtatccttgaatcgaaacctttttctgagtatttaacaatcgcaccctttaaaaaatgtacatagacattaagagacttaaacagatatataatcattttaaattaaaatagcgttaaacagtacctcaagctcaataagcattttaagtattctaatcttagtatttctctagctgacatgtaagaagcaatctatcttattgtatgcaattagctctttgtgtggataaaaaggtaaaaccattctgaaacaggaaaccaatacacttcctgtttaatcaacaaatctaaacatttattcttttcatctgtttactcttgctcttgtccaccacaatatgctattcacatgttcagtgtagttttatgacaaagaaaattttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagactggtaaagtctttgaaaatatgtgtaatatgtaaaacattttgacacccccataatatttttccagaattaacagtataaattgcatctcttgttcaagagttccctatcactctttaatcactactcacagtaacctcaactcctgccacaatgtacaggatgcaactcctgt

SEQ ID NO: 30 - DNA, 30 bp, Artificial Sequence
  PCR primer for amplifying the 5′-flanking region of Human interleukin-2 (IL-2) gene
cgctagctgctcttgtccaccacaatatgc

SEQ ID NO: 31 - DNA, 538 bp, Homo sapiens
  misc_feature (1)..(538): Corresponding to the sequence (+901 to +1438) in the GenBank database (Acession HSIL05)
taaaaccattctgaaacaggaaaccaatacacttcctgtttaatcaacaaatctaaacatttattcttttcatctgtttactcttgctcttgtccaccacaatatgctattcacatgttcagtgtagttttatgacaaagaaaattttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagactggtaaagtctttgaaaatatgtgtaatatgtaaaacattttgacacccccataatatttttccagaattaacagtataaattgcatctcttgttcaagagttccctatcactctttaatcactactcacagtaacctcaactcctgccacaatgtacaggatgcaactcctgtc

SEQ ID NO: 32 - DNA, 28 bp, Artificial Sequence
  PCR primer for amplifying the promoter region of Human GATA-1 gene
atagatctatccctggctcccacctcag

SEQ ID NO: 33 - DNA, 28 bp, Artificial Sequence
  PCR primer for amplifying the promoter region of Human GATA-1 gene
ataagctttggtggttgcggagggttcg

SEQ ID NO: 34 - DNA, 28 bp, Artificial Sequence
  PCR primer for amplifying the promoter region of Human GATA-1 gene
atggtaccaccccagaagatgccaggag

SEQ ID NO: 35 - DNA, 28 bp, Artificial Sequence
  PCR primer for amplifying the promoter region of Human GATA-1 gene
atgctagcgccctctgagcctcagtttc

SEQ ID NO: 36 - DNA, 23 bp, Artificial Sequence
  PCR primer for amplifying a human histone deacetylase-1 (HDAC1) gene
gaggaattcaagatggcgcagac

SEQ ID NO: 37 - DNA, 25 bp, Artificial Sequence
  PCR primer for amplifying a human histone deacetylase-1 (HDAC1) gene
ggagcggccgcttcaggccaacttg

SEQ ID NO: 38 - DNA, 24 bp, Artificial Sequence
  PCR primer for amplifying a human histone deacetylase-2 (HDAC2) gene
ggggatccatggcgtacagtcaag

SEQ ID NO: 39 - DNA, 29 bp, Artificial Sequence
  PCR primer for amplifying a human histone deacetylase-2 (HDAC2) gene
ggtgcggccgccaaattcaggggttgctg

SEQ ID NO: 40 - DNA, 22 bp, Artificial Sequence
  PCR primer for amplifying a human histone deacetylase-3 (HDAC3) gene
ccggatccaccatggccaagac

SEQ ID NO: 41 - DNA, 28 bp, Artificial Sequence
  PCR primer for amplifying a human histone deacetylase-3 (HDAC3) gene
gcagcggccgccactcttaaatctccac

SEQ ID NO: 42 - DNA, 28 bp, Artificial Sequence
  PCR primer for amplifying a human histone deacetylase-4 (HDAC4) gene
gggaattcatgagctcccaaagccatcc

SEQ ID NO: 43 - DNA, 29 bp, Artificial Sequence
  PCR primer for amplifying a human histone deacetylase-4 (HDAC4) gene
aaggcgccgcagcttcgagggagtgctac

SEQ ID NO: 44 - DNA, 27 bp, Artificial Sequence
  PCR primer for amplifying a human histone deacetylase-5 (HDAC5) gene
ggcaagcttatgaactctcccaacgag

SEQ ID NO: 45 - DNA, 29 bp, Artificial Sequence
  PCR primer for amplifying a human histone deacetylase-5 (HDAC5) gene
gggcggccgcgtcacagggcaggctcctg

SEQ ID NO: 46 - DNA, 27 bp, Artificial Sequence
  PCR primer for amplifying a human histone deacetylase-6 (HDAC6) gene
gcgaagcttatgacctcaaccggccag

SEQ ID NO: 47 - DNA, 33 bp, Artificial Sequence
  PCR primer for amplifying a human histone deacetylase-6 (HDAC6) gene
gcatgcggccgcttagtgtgggtggggcatatc

SEQ ID NO: 48 - DNA, 26 bp, Artificial Sequence
  PCR primer for amplifying a human histone deacetylase-7 (HDAC7) gene
cgaattcagccgcagcccatggacct

SEQ ID NO: 49 - DNA, 26 bp, Artificial Sequence
  PCR primer for amplifying a human histone deacetylase-7 (HDAC7) gene
cggtaccctgtgcacccggatcacgg

SEQ ID NO: 50 - DNA, 26 bp, Artificial Sequence
  PCR primer for amplifying a human histone deacetylase-8 (HDAC8) gene
cgaattctttaagcggaagatggagg

SEQ ID NO: 51 - DNA, 26 bp, Artificial Sequence
  PCR primer for amplifying a human histone deacetylase-8 (HDAC8) gene
aggtaccgaccacatgcttcagattc

SEQ ID NO: 52 - DNA, 24 bp, Artificial Sequence
  PCR primer for amplifying a human histone deacetylase-3 (HDAC3) gene
ctgaattcaccatggccaagaccg

SEQ ID NO: 53 - DNA, 61 bp, Artificial Sequence
  PCR primer for adding a Flag sequence to a human histone deacetylase-3 (HDAC3) gene
gggcggccgcctacttgtcatcgtcgtccttgtaatcggtaccaatctccacatcgctttc

SEQ ID NO: 54 - protein, 8 aa, Artificial Sequence
  Flag sequence
DYKDDDDK

SEQ ID NO: 55 - DNA, 31 bp, Artificial Sequence
  PCR primer for adding a Kpn I site to a human histone deacetylase-1 (HDAC1) gene
atcggtaccggccaacttgacctcctccttg

SEQ ID NO: 56 - DNA, 29 bp, Artificial Sequence
  PCR primer for adding a Kpn I site to a human histone deacetylase-2 (HDAC2) gene
atcggtaccggggttgctgagctgttctg

SEQ ID NO: 57 - DNA, 27 bp, Artificial Sequence
  PCR primer for adding a Bgl II site to a human histone deacetylase-4 (HDAC4) gene
gatagatctcaggggcggctcctcttc

SEQ ID NO: 58 - DNA, 46 bp, Artificial Sequence
  Nucleotide sequence having a Flag nucleotide sequence
aattcctgcagagatctgattacaaggacgacgatgacaagtaggc

SEQ ID NO: 59 - DNA, 46 bp, Artificial Sequence
  Nucleotide sequence having a Flag nucleotide sequence
ggccgcctacttgtcatcgtcgtccttgtaatcagatctctgcagg

SEQ ID NO: 60 - DNA, 23 bp, Artificial Sequence
  PCR primer for creating a dominant negative mutant (H802K, H803L) of human histone deacetylase-4 (HDAC4) gene
cagtgacaccatatggaacgagg

SEQ ID NO: 61 - DNA, 20 bp, Artificial Sequence
  PCR primer for creating a dominant negative mutant (H802K, H803L) of human histone deacetylase-4 (HDAC4) gene
gcaagctttccaggggggcg

SEQ ID NO: 62 - DNA, 20 bp, Artificial Sequence
  PCR primer for creating a dominant negative mutant (H802K, H803L) of human histone deacetylase-4 (HDAC4) gene
ccccctggaaagcttgcgga

SEQ ID NO: 63 - DNA, 22 bp, Artificial Sequence
  PCR primer for creating a dominant negative mutant (H802K, H803L) of human histone deacetylase-4 (HDAC4) gene
gacatgtacaggacgctagggt

SEQ ID NO: 64 - DNA, 21 bp, Artificial Sequence
  PCR primer for creating a dominant negative mutant (H863L) of human histone deacetylase-4 (HDAC4) gene
cctgtacatgtccctccttcg

SEQ ID NO: 65 - DNA, 20 bp, Artificial Sequence
  PCR primer for creating a dominant negative mutant (H863L) of human histone deacetylase-4 (HDAC4) gene
atagatctagacaggggcgg

SEQ ID NO: 66 - DNA, 23 bp, Artificial Sequence
  PCR primer for creating a dominant negative mutant (H140K, H141L) of human histone deacetylase-1 (HDAC1) gene
gaggaattcaagatggcgcagac

SEQ ID NO: 67 - DNA, 25 bp, Artificial Sequence
  PCR primer for creating a dominant negative mutant (H140K, H141L) of human histone deacetylase-1 (HDAC1) gene
ctcggacttctttgcaagcttcagg

SEQ ID NO: 68 - DNA, 24 bp, Artificial Sequence
  PCR primer for creating a dominant negative mutant (H140K, H141L) of human histone deacetylase-1 (HDAC1) gene
gggcctgaagcttgcaaagaagtc

SEQ ID NO: 69 - DNA, 25 bp, Artificial Sequence
  PCR primer for creating a dominant negative mutant (H140K, H141L) of human histone deacetylase-1 (HDAC1) gene
gtgaatatcaatgtcaatgtacagc

SEQ ID NO: 70 - DNA, 21 bp, Artificial Sequence
  PCR primer for creating a dominant negative mutant (H199L) of human histone deacetylase-1 (HDAC1) gene
gacggcgtggaagaggccttc

SEQ ID NO: 71 - DNA, 26 bp, Artificial Sequence
  PCR primer for creating a dominant negative mutant (H199L) of human histone deacetylase-1 (HDAC1) gene
gaagtactctccatacttaagaaagg

SEQ ID NO: 72 - DNA, 26 bp, Artificial Sequence
  PCR primer for creating a dominant negative mutant (H199L) of human histone deacetylase-1 (HDAC1) gene
ctgtgtcctttcttaagtatggagag

SEQ ID NO: 73 - DNA, 22 bp, Artificial Sequence
  PCR primer for creating a dominant negative mutant (H199L) of human histone deacetylase-1 (HDAC1) gene
tctcggagcgggtagttaacag

SEQ ID NO: 74 - DNA, 24 bp, Artificial Sequence
  PCR primer for creating a dominant negative mutant (H134K, H135L) of human histone deacetylase-3 (HDAC3) gene
aacccagctgaacaacaagatctg

SEQ ID NO: 75 - DNA, 26 bp, Artificial Sequence
  PCR primer for creating a dominant negative mutant (H134K, H135L) of human histone deacetylase-3 (HDAC3) gene
ctcaaacttcttggcaagcttcagac

SEQ ID NO: 76 - DNA, 25 bp, Artificial Sequence
  PCR primer for creating a dominant negative mutant (H134K, H135L) of human histone deacetylase-3 (HDAC3) gene
tggtggtctgaagcttgccaagaag

SEQ ID NO: 77 - DNA, 21 bp, Artificial Sequence
  PCR primer for creating a dominant negative mutant (H134K, H135L) of human histone deacetylase-3 (HDAC3) gene
cccgtcaccatggtggatgtc

SEQ ID NO: 78 - DNA, 23 bp, Artificial Sequence
  PCR primer for creating a dominant negative mutant (H193L) of human histone deacetylase-3 (HDAC3) gene
tgacattgacatccaccatggtg

SEQ ID NO: 79 - DNA, 26 bp, Artificial Sequence
  PCR primer for creating a dominant negative mutant (H193L) of human histone deacetylase-3 (HDAC3) gene
gaagtaatttccgtacttaaggaagg

SEQ ID NO: 80 - DNA, 26 bp, Artificial Sequence
  PCR primer for creating a dominant negative mutant (H193L) of human histone deacetylase-3 (HDAC3) gene
tgtccttccttaagtacggaaattac

SEQ ID NO: 81 - DNA, 22 bp, Artificial Sequence
  PCR primer for creating a dominant negative mutant (H193L) of human histone deacetylase-3 (HDAC3) gene
gagcacaatgcacgtgggttgg

SEQ ID NO: 82 - DNA, 29 bp, Artificial Sequence
  PCR primer for amplifying RD3 sequence of human N-CoR gene
gggatccgtgaagtccttcagcctgctcc

SEQ ID NO: 83 - DNA, 29 bp, Artificial Sequence
  PCR primer for amplifying RD3 sequence of human N-CoR gene
tgcggccgccatgggtgagcctctggaca

SEQ ID NO: 84 - DNA, 960 bp, homo sapiens
  misc_feature (1)..(960): Corresponding to the sequence (+5281 to +6240) in the GenBank database (Acession AF196971)
gcctctgtctctcccactccacccctttcctttcctaccctatcccactcctcgaggaatcatccctggctcccacctcagtttcccgcctccaaggcagcatggcgggcaagaagttgaggccactgtccctgggtgttcctacccccacaccctcaccccaagacagcctgttactgcggcgccaacagccacggtcgcctacatctgataagacttatctgctgccccagggcaggccggagctggcgtaagccccagtggggcgctaagtgagtgtgcccctgcctcccgccagcactggcctggcctgcaggcttagcctgggtcatcaaggtatcccacaggctctagttcaaatccagcagaacctctctgagcctcactcttctcacctgcaaaatgggtacagccacatcccttctctccctgcagccaggaagacgcacatacacaggagtctagcccacaccggccccgcacaaattaagggctttactctctgaaaagcccagtgaagtcatgaaaccatatctgctattttcatttatcttggtttcagcctattttgcttgtctggacactacagtccacgggagcctaggtcgagcgaggtccaagaatccccagggtgggcagggagggtggaagagggcctccagtgcccaagaggtgccccacaagcatgggacccgccccctcccctggactgccccacccactggggcaccagccactccctggggaggagggaggagggagaagggagggagggagggagggaggaagggagcctcaaaggccaaggccagccaggacaccccctgggatcacactgagcttgccacatccccaaggcggccgaaccctccgcaaccaccagcccaggtcagtctcagcccccagagagcccccaccaaggcaaccctgggcctgctgcccctacccttatgcttgctc

SEQ ID NO: 85 - DNA, 720 bp, homo sapiens
  misc_feature (1)..(720): Corresponding to the sequence (+2321 to +3040) in the GenBank database (Acession AF196971)
gtacaaatgccatggggcctggaccaagcaggggctctagaaccccagaagatgccaggagggagtgagccagtcagggaaggcttccgagaagagaggacattgaagaagagtctcaaacttaggcctgacggagaagacgcgcggccaggacaccccacccccgccctcgtctcccccaaagcctgatctggccccactgattcccttatctgcccactcccagctgcctccttgctggctgaactgtcgccgcagacttctgagcctgcgccccctccacggggatgggggagggaatggggtgaggcctggcctcacagcctcggggtttccagctcttgctggaggcagggctctggggcgccctactcctcacccttggcttctcttcctgagcgctctgtgctctccagaaatgaagaaatggggtgagtccagcggccaaacccttgtcttagctcttagacatgcctcgagcctgccattccctgtgaggacagatttccctatgttgcgaccgctgcttctaataataataatgatgatgataattcccatttacagagcacaccatttatggtgtgccagcaggccctgtgctgagtggttcctacccacgtggggggctaggactttacccgttttccagatgaagaaactgaggctcagagggcgtctggcccaggaatcacacagcaaatcacacagcaaatcaga

SEQ ID NO: 86 - DNA, 12 bp, homo sapiens
  misc_feature (1)..(12): Palindrome sequence founed on the 3′ side of the GATA-E-box motif in promoter reagion of human GATA-1 gene
ctgtggccacag